SEQUENCE LISTING (34 sequences)

SEQ ID NO: 1 - protein, 599 aa, Trichoderma reesei
SVDDFISTETPIALNNLLCNVGPDGCRAFGTSAGAVIASPSTIDPDYYYMWTRDSALVFKNLIDRFTETYDAGLQRRIEQYITAQVTLQGLSNPSGSLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSALHLTWSYASFLTATARRAGIVPPSWANSSASTIPSTCSGASVVGSYSRPTATSFPPSQTPKPGVPSGTPYTPLPCATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 2 - protein, 619 aa, Aspergillus kawachii
LSAAEWRTQSIYFLLTDRFGRTDNSTTATCNTGDQIYCGGSWQGIINHLDYIQGMGFTAIWISPITEQLPQDTSDGEAYHGYWQQKIYNVNSNFGTADDLKSLSDALHARGMYLMVDVVPNHMGYAGNGNDVDYSVFDPFDSSSYFHPYCLITDWDNLTMVQDCWEGDTIVSLPDLNTTETAVRTIWYDWVADLVSNYSVDGLRIDSVEEVEPDFFPGYQEAAGVYCVGEVDNGNPALDCPYQKYLDGVLNYPIYWQLLYAFESSSGSISNLYNMIKSVASDCSDPTLLGNFIENHDNPRFASYTSDYSQAKNVLSYIFLSDGIPIVYAGEEQHYSGGDVPYNREATWLSGYDTSAELYTWIATTNAIRKLAISADSDYITYANDPIYTDSNTIAMRKGTSGSQIITVLSNKGSSGSSYTLTLSGSGYTSGTKLIEAYTCTSVTVDSNGDIPVPMASGLPRVLLPASVVDSSSLCGGSGNTTTTTTAATSTSKATTSSSSSSAAATTSSSCTATSTTLPITFEELVTTTYGEEVYLSGSISQLGEWDTSDAVKLSADDYTSSNPEWSVTVSLPVGTTFEYKFIKVDEGGSVTWESDPNREYTVPECGSGSGETVVDTWR

SEQ ID NO: 3 - protein, 616 aa, Aspergillus terreus
APQLAPRATTSLDAWLASETTVALDGILDNVGSSGAYAKSAKSGIVIASPSTSDPDYYYTWTRDAALTVKALIDLFRNGETSLQTVIMEYISSQAYLQTVSNPSGSLSTGGLAEPKYYVDETAYTGSWGRPQRDGPALRATAMIDFGNWLIDNGYSTYASSIVWPIVRNDLSYVAQYWNQTGYDLWEEVNGSSFFTIAVQHRALVEGSTFASKVGASCSWCDSQAPQVLCFLQRFWTGSYIMANFGGGRSGKDANTVLGSIHTFDPNAGCDDTTFQPCSPRALANHKVYTDSFRSIYSINSGISSGKAVAVGRYPEDSYYNGNPWFLTTLAAAEQLYDAIYQWQKIGSITITDVSLAFFKDLYSSAAVGTYASSSSAFTSIVSAVKTYADGYMSIVQTHAMTNGSLSEQFGKSDGFSLSARDLTWSYAALLTANLRRNSVVPPSWGETTATSVPSVCSATSATGTYSTATNTAWPSTLTSGTGATTTTSKATSSSTTTTSSASSTTVECVVPTAVAVTFDEVATTTYGENVYVVGSISQLGSWDTSKAVALSASKYTSSNNLWYVTVTLPAGTTFQYKFIRVSSSGSVTWESDPNRSYTVPSACGTSTAVVNTTWR

SEQ ID NO: 4 - protein, 612 aa, Aspergillus fumigatus
APQLSARATGSLDSWLGTETTVALNGILANIGADGAYAKSAKPGIIIASPSTSEPDYYYTWTRDAALVTKVLVDLFRNGNLGLQKVITEYVNSQAYLQTVSNPSGGLASGGLAEPKYNVDMTAFTGAWGRPQRDGPALRATALIDFGNWLIDNGYSSYAVNNIWPIVRNDLSYVSQYWSQSGFDLWEEVNSMSFFTVAVQHRALVEGSTFAKRVGASCSWCDSQAPQILCYMQSFWTGSYINANTGGGRSGKDANTVLASIHTFDPEAGCDDTTFQPCSPRALANHKVYTDSFRSVYAINSGIPQGAAVSAGRYPEDVYYNGNPWFLTTLAAAEQLYDAIYQWKKIGSISITSTSLAFFKDIYSSAAVGTYASSTSTFTDIINAVKTYADGYVSIVQAHAMNNGSLSEQFDKSSGLSLSARDLTWSYAAFLTANMRRNGVVPAPWGAASANSVPSSCSMGSATGTYSTATATSWPSTLTSGSPGSTTTVGTTTSTTSGTAAETACATPTAVAVTFNEIATTTYGENVYIVGSISELGNWDTSKAVALSASKYTSSNNLWYVSVTLPAGTTFEYKYIRKESDGSIVWESDPNRSYTVPAACGVSTATENDTWQ

SEQ ID NO: 5 - protein, 608 aa, Neosartorya fischeri
APQLSPRATGSLDSWLATESTVSLNGILANIGADGAYAKSAKPGIIIASPSTSDPDYYYTWTRDAALVTKVLVDLFRNGNLGLQKVITEYVNSQAYLQTVSTPSGGLSSGGLAEPKYNVDMTAFTGAWGRPQRDGPALRATALIDFGNWLIDNGYSSYAVNNIWPIVRNDLSYVSQYWSQSGFDLWEEVNSMSFFTVAVQHRALVEGSTFAKRVGASCSWCDSQAPQILCYMQSFWTGSYINANTGGGRSGKDANTVLASIHTFDPEAGCDDTTFQPCSPRALANHKVYTDSFRSVYAINSGIPQGVAVSAGRYPEDVYYNGNPWFLTTLAAAEQLYDAIYQWKKIGSISITSTSLAFFKDIYSSVAVGTYASSSSTFTAIIDAVKTYADGYVSIVEAHAMTNGSLSEQFDKSSGMSLSARDLTWSYAALLTANMRRNGVVPAPWGAASANSVPSSCSMGSATGTYSTATATSWPSTLTSGSPSDTTSGTTPGTTTTTSACTTPTSVAVTFDEIATTTYGENVYIIGSISQLGSWDTSKAVPLSSSKYTSSNNLWYVTINLPAGTTFEYKYIRKESDGSIEWESDPNRSYTVPSACGVSTATEKDTWR

SEQ ID NO: 6 - protein, 612 aa, Neosartorya fumigata
APQLSARATGSLDSWLGTETTVALNGILANIGADGAYAKSAKPGIIIASPSTSEPDYYYTWTRDAALVTKVLVDLFRNGNLGLQKVITEYVNSQAYLQTVSNPSGGLASGGLAEPKYNVDMTAFTGAWGRPQRDGPALRATALIDFGNWLIDNGYSSYAVNNIWPIVRNDLSYVSQYWSQSGFDLWEEVNSMSFFTVAVQHRALVEGSTFAKRVGASCSWCDSQAPQILCYMQSFWTGSYINANTGGGRSGKDANTVLASIHTFDPEAGCDDTTFQPCSPRALANHKVYTDSFRSVYAINSGIPQGAAVSAGRYPEDVYYNGNPWFLTTLAAAEQLYDAIYQWKKIGSISITSTSLAFFKDIYSSAAVGTYASSTSTFTDIINAVKTYADGYVSIVQAHAMNNGSLSEQFDKSSGLSLSARDLTWSYAAFLTANMRRNGVVPAPWGAASANSVPSSCSMGSATGTYSTATATSWPSTLTSGSPGSTTTVGTTTSTTSGTATETACATPTAVAVTFNEIATTTYGENVYIVGSISELGNWDTSKAVALSASKYTSSNNLWYVSVTLPAGTTFEYKYIRKESDGSIVWESDPNRSYTVPAACGVSTATENDTWR

SEQ ID NO: 7 - protein, 618 aa, Penicillium marneffei
APQFSPRATVGLDAWLASETTFSLNGILANIGSSGAYSASAKPGVVIASPSTNNPNYYYTWTRDSALTLKVLIDLFGNGNLSLQTVIEEYINAQAYLQTVSNPSGDLSSGAGLAEPKYNVDMSPFTGGWGRPQRDGPALRAIALIEFGNWLIDNGYSSYAVNNIWPIVRNDLSYVSQYWSQSGFDLWEEVNSMSFFTVANQHRALVQGSTFAARVGASCSWCDSQAPQILCYMQTFWTGSYINANTGGGRSGKDSNTVLTTIHTFDPEATCDDVTFQPCSPRALANHKVYTDSFRSIYGVNSGIAQGVAVSVGRYPEDSYYGGNPWFLSNLAAAEQLYDAIYQWNKIGSITITSTSLAFFKDVYSSAAVGTYASGSTAFTSIISAVKTYADGYVSIVQGHAAANGSLSEQFDRNSGVEISARDLTWSYAALLTANLRRNGVMPPSWGAASANSVPSSCSMGSATGTYSTPTATAWPSTLTSATGIPVTTSATASVTKATSATSTTTSATTCTTPTSVAVTFDEIATTTYGENVFIVGSISQLGSWDTSKAIALSASQYTSSNHLWFATLSLPAGTTFQYKYIRKESNGSIVWESDPNRSYTVPSGCGVSTATENDTWR

SEQ ID NO: 8 - protein, 617 aa, Talaromyces stipitatus
APGLSPRASTSLDAWLATETTVSLSGILANIGADGAYSKSAKPGVVIASPSTDNPNYYYTWTRDSALTLKVLIDLFRNGNLGLQTVIEEYVNAQAYLQTVSNPSGDLSSGAGLAEPKFNVDMSAFTGSWGRPQRDGPALRAIALIDFGNWLIENGYTSLAANNIWPIVRNDLSYVAQYWSQSGFDLWEEVNSMSFFTVANQHRSLVEGSTFAAKVGASCSWCDSQAPQILCYMQTFWTGSYMNANTGGGRSGKDANTVLTSIATFDPEATCDDVTFQPCSPRALANHKVYTDSFRSVYGLNSGIAEGVAVAVGRYPEDSYYNGNPWFLSNLAAAEQLYDAIYQWNKIGSITITSTSLAFFKDVYSSAAVGTYASGSSAFTSIINAVKTYADGYISVVQSHAMNNGSLSEQFDKNTGAELSARDLTWSYAALLTANMRRNGVVPPSWGAASATSIPSSCTTGSAIGTYSTPTATSWPSTLTSGTGSPGSTTSATGSVSTSVSATTTSAGSCTTPTSVAVTFDEIATTSYGENVYIVGSISQLGSWNTANAIALSASKYTTSNNLWYVTINLPAGTTFQYKYIRKESDGTVKWESDPNRSYTVPSACGVSTATENDTWR

SEQ ID NO: 9 - protein, 560 aa, Metarhizium acridum
HRDDLHGFITKQKSISLHGVLANIGSDGSRAQGAAAGAVVASPSKSDPDYWYTWSRDSALTFKVLIELFIGGKKSLQPKIEQYITAQAHLQGVSNPSGGPDTGGLGEPKFHVNLTAFTGSWGRPQRDGPPLRATALTIYANWLIANGGQAEAANTVWPIIAKDLSYTVQYWNRTGFDLWEEINGSSFFTLSASFRALVEGATLAKALGKQCPDCETNAPRILCFLQSFWANGYIDSNINVNDGRTGKDVNSIISSIHTFDPAAACTDATFQPCSSRALANHKAVVDSFRTIYTVNKGRRPGRAAAVGRYSEDVYYNGNPWYLATMAAAEQMYAAVYQWREIGSITVDATSLPFFSDLIPNIAAGTYAKNSATFTSIIKAATAYGDDFVRVVKQYTPADGSLAEQYDRETGSPKSAVHLTWSYASFVGAVERRSGIVPPSWGEPNSNTVPKVCEAPPSCDSTMTFNVKVTTVPGESIYVVGSITELKNWSPADAVPLDASQYTPSNPLWSAKVTIPAGTNFEYKYIKKTSDGTVVWESDPNRSATSSTGCQSNGTLNDQWR

SEQ ID NO: 10 - protein, 556 aa, Schizophyllum commune
QTSAADAYVSAESPIAQAGILANIGPSGSKSHGAASGVIIASPSTSNPDYLYTWTRDAALVSRALVDEFIEGESSLQSVIDSYVSSQQKLQRVDNPSGSYTSGGLGEPKFNIDLTAFTGAWGRPQRDGPALRAITLITYGNHLLSSGNTSYVTDTIWPVVKADLDYVVSYWNQTGFDLWEEVSSSSFFTTAEQHTALRLGATFATAVGASASTYLTQADNVLCFLQSYWNSNGGYATANTGGGRSGIDANTVLTSIHTFDIEAGCDSVTFQPCSDRALSNLKVYVDSFRGLYSINPTGATDPILTGRYKEDVYYNGNPWYLTTFAVAEQLYDALNTWDKLGSLDVTSTSLAFFKQFDSSITAGTYASSTSEYATLTSAIRNWADGFLEVLADFTPADGGLTEQIDKSSGNPTSAADLTWSYASAITAFKARGGAIPASWGAAGLTVPATCSTGGGGGSGGDTVAVTLNVQATTVYGENIYVTGSVNQLANWSPDNAIALNADNYPTWSVTVNLPANTQIEYKYIRKNNGQVTWESDPNRSITTSASGSFTQNDTWR

SEQ ID NO: 11 - protein, 627 aa, Trichoderma atroviridis
VPRLRESRHEFDIVKRSASSFLETEVPIALADLLCNIGSAGSCAAGANSGIVIASPSKTNPDYFYTWTRDSALVFKCIVDTFVNSYSASLQTEIENYINAQAIVQGISNPSGSLSNSGTGLGEPKFNVDETAFTGAWGRPQRDGPALRAIALITYSKWLINNGYQSTANSIVWPIIQNDISYVAQYWNQTGFDLWEEVNGSSFFTVANQHRALVEASALATSLGKSLPNASSQAAQALCFLQSFWSSSQGYIVANINQNNGRSGKDANTLLGSIHTFDPEGNCDASTFQPCSDRTLANHKVVVDSFRSIYTINNGIPAGTAAAVGRYPEDSYQGGNPWYLNTLAAAELLYDALYQWKRIGAITVTSTSLAFFKDLDSSITVGTYSSSSSTYTTLYNAVSNYADGFVNNVATYAPSNGSLAEQYNRNNGQPLSAYDLTWSYAALLTAAARRSGVVPYSWGETSASSVPSVCSYTSAVGSYSSASTGSWPPNQTPTDGSGSTTSKSTSVTVSSTSTSASSTAVATSPVTVTFDEIVTTIFGQTIKIAGNVPVLGNWNTNNAVALSADGYTSSNHLWNVGISFAPGTVIQYKYINVASNGDVTWEADPNHTYTVPATGATAVTVNNSWQS

SEQ ID NO: 12 - protein, 561 aa, Bjerkandera adusta
QSSTVDAFIASESPIARTGLLANIGADGSKASGAKSGIVIASPSKSNPDYFYTWTRDAALVFKAIIDRYTSGEDTATRRQIDEYVSGQALLQQVSNPSGTVSTGGLAEPKYNVDMSAFTGGWGRPQRDGPALRATAIIAYANWLVANGNTSYVTSTLWPVLKLDLDYVRDNWNQTGFDLWEEINSSSFFTTAVQHRALREGNALAAKIGQTVSGYTTQADNVLCFLQSYWNPSGGFATSNTGGGRSGKDANSVLTSIHTFDAAAGCDALTFQPCSDRALSNHKVYVDSFRSIYSVNSGIASNAAVATGRYPEDSYYGGNPWYLTTLAAAEQLYDALTVWDAQGSLNVTSVSLAFFQQFAPTVTAGTYPASSATYGTLTAAIRAYADGFVAVVAKYTPSNGGLAEQYTRAGGTPTSAADLTWSYAAALTAFSAREGFTPASWGAKGLTAPAACNTNSGGGSGGGSGNTVAVTFNVQATTVWGENIYLTGSVDALQNWSPDNALLLSSANYPTWSITVNLPPSTAIQYKYIRKNNGAVTWESDPNMSITTPGSGSATLSDTWR

SEQ ID NO: 13 - protein, 558 aa, unidentified
QSSADAYVASEASIAKAGLLANIGANGSKSEGAKAGIVVASPSTSNPDYLYTWTRDSSLVFKTVIDQFTTGEDTSLRGLIDEFTAAQSILQQTSNPSGSVSTGGLGEPKFNVDETAFTGAWGRPQRDGPALRATAIITYANWLLANGNGTSYVQNTLWPIIKLDLDYVENNWNQSTFDLWEEVNSSSFFTTAVQHRALREGVALASAIGQTSVVSGYSAQADNLLCFLQSYWNSGSGFVTANTGGGRSGRDANTVLTSIHTFDVEAGCDAVTFQPCSDKALSNLKVYVDAFRSIYGINSGIASNAAVATGRYPEDSYYNGNPWYLAVFAVAEQLYDALITWDELGSLNVTSTSLAFFQQFDSSVTAGTYDSSSSTYSTLTSGIKGFADGFLEVNSKYTPSTGALSEQFDKSSGSQLSASDLTWSYAAALTAFAARSGKTYASWGAAGLITTCGGSGGGGGGSGTVSVTFNVQATTVFGENIYITGSVDALQNWSPDNALILSAANYPIWSITVSLPASTVIEYKYIRKFNGQVTWESDPNDSITTPASGSYIENDTWR

SEQ ID NO: 14 - protein, 562 aa, Termetes versicolor
QSSVADAYVASESSIAKAGVLANIGPSGSKSQGAKAGIVVASPSTTNPDYLFTWTRDTSLVFKALIDQLTSGEDPSLRGLVDMFTSSQAALQQVSNPSGTVSTGGLGEPKFNIDESAFTGAWGRPQRDGPALRSTAFISYANWLLDNGNTTYVTQTLWPVIKLDLDYVEANWNQTGFDLWEEVNSSSFFTTAVQHRALREGAAFATRIGQTSVVSGYTTQAANVLCFLQSYWNPSGGFVTANTGGGRSGRDANTVLTSIHTFDPAAGCDATTFQPCSDKALSNLKVYVDAFRSIYTINSGIAANAAVATGRYPEDSYQGGNPWYLATIAVAEQLYDALIVWDQLGSINVTTTSLPFFQQFSSTVTTGTFASTSATYTTLTTAVRNFADGFIAVNAQFTPSNGGLAEQFSRSNGQPVSAVDLTWSYAATLTAFHARAGLTYPGWGAAGLTVPAVCSTSGSGSGGGGAGTVAVTFNVQATTFFGENIYITGSVDALQNWSPDNALLLSSANYPIWSITVNLPASTSVQYKFIRKAPGELIWESDPNNQITTPASGTFTQSDTFR

SEQ ID NO: 15 - protein, 563 aa, Hypholoma sublateritium
QSSAVSSYLATESVIAKAGLLANIGPSGSKASGAVSGVVVAAPSTNPDYIFTWTRDSALVFKAIIDSFARGEDATLRTSIDQYVAAQKIQQQVSNPSGTVSSGGLGEPKFNVDLSAFTGAWGRPQRDGPALRATALISYGNSLISASNTSYVLANIWPLVKLDLDYVAANWNQTGFDLWEEVNSSSFFTTAVQHRALRQGAAFATALGQTASVAGYTTQAANVLCFLQSYWNPSQGYITANTGGGRSGKDANTALASIHTFDPTAGCDAATFQPCSDKALSSLKVYVDSFRSIYTVNSAVASPGAVATGRYPEDSYFGGNPWYLATMAVAEQLYDALIVWKAQGSLNVTSTSLAFFQQFSSAVTVGTYASTTATFTTLTTAIANQADGFVAIVQEFTPSTGSLSEQYSRSNGAQLSANDLTWSYASILTAVTARNGLAGDNWGAAGLVVPSSCSTSGTGSSSGGGSSGTVAVTFKVTATTTFGENIYLTGSDDALEDWSPTSTLILSAATYPVWSITVNLPASTALQYKYIRIFNGVTTWESDPNNAFTTVASGTQTLTDTWR

SEQ ID NO: 16 - protein, 559 aa, Fomitiporia mediterranea
QTAVDSYVATESPIAKTNLLANIGASGSKSQGAKPGIVIASPSTTNPNYLFTWTRDSSLVFKTIIDQYTNGQDTSLRTLIDEFVSAEATLQQVSNPSGTVSTGGLGEPKFNIDETAFTGAWGRPQRDGPALRATAIINYANYLLANDNSSFVTNTLWPILQLDLDYVAQDWNQTTFDLWEEVDSTSFFTAAVQHRSLREGATLATKIGQTSVVSGYTTQAENILCFMQSFWNAGGNFMTANTGGGRSGKDANTVLASIHTFDSSAGCDAATFQPCSDRALANLKTYVDAFRSIYSINSGIASNAAVATGRYPEDVYFNGNPWYLTTLSVAEQLYDAITVWNAQGSLNVTSVSQPFFALFQSDIAVGTYASSSSTFTSLLSSIKSFADGFVSVVAKYTPSNGGLSEQYSKSDGTPTSAVDLTWSYAAALTAFAARDGFVPASWGAAGLTVPSTCSTSGSGPGSGGTVAVTFNVQATTVFGENIYITGSVDALQNWSPDNAIILSAANYPTWSVTINLPASTTIQYKYIRKFNGAVTWESDPNMQITTPSGGTFIENDVWR

SEQ ID NO: 17 - protein, 562 aa, Punctularia strigosozonata
QTASAAAYATTEAPIAKAGVLANIGPSGSKSQGAKAGIVIASPSTSNPDYLYTWTRDSSLVFKALIDQYTSGIDTTLRGAIDNFFNAEKILQQVSNPSGTVSTGGLGEPKFNIDETAFTGAWGRPQRDGPALRATALITYANYLYSTGNTTFVSNTLWPVIKLDLDYAANNWNQTTFDLWEEVSSSSFFTTAVQHRSLREGATLATKLGVTSSASTYTSAASSLLCFLQSYWNPAGGYITANTGGGRSGKDANTVLTSIHTFDPAAGCDAVTFQPCSDKALSNLKVYVDSFRSIYGINSGIASNAAVATGRYPEDTYYNGNPWYLTTLAVAEQLYDALIVWNARGSLNVTSTSLAFFQQFSSSVTTGTYPSTSTTFTTLTSAVKTFADGFVAVVAKYTPSSGALSEQFDKSSGSQLSAADLTWSYAAALTAFEARNGTTFASWGAAGLTTSCSSSGSGSGGGSGSSGSVPVNFQETATTVYGENIFIVGSISPLGNWDPNSAIALSAANYPNWQVSISLPASTTFQYKYIRKYNGAVTWESDPNRSFTTPSSGSYNENDTWR

SEQ ID NO: 18 - protein, 554 aa, Phlebia brevispora Nakasone
QTNVNSYVASESAFAKAGLLANIGPSGSKSSGADPGIVIASPSTTNPDYLYTWVRDSSLVFKVLIDQYTTGVDTSLRTLIDEFVSAEAILQQVTNPSGSVTTGGLGEPKFNIDETAFTGSWGRPQRDGPALRSTAIITYANWLLDNGNTSYVTETLWPVLELDLNYVMNNWNQSTYDLWEEIDSSSFWTTAVQHRALRQGSALATRIGQTSMVSGYNTQAANVLCFLQSFWNPSGNYVTANTGGGRSGIDANTVLTSIHTFDPSAGCDATTFQPCSDKALANLKVYTDSFRSIYSVNTGIASNAAVATGRYPEDVYMGGNPWYLATMAAAEQLYDALSVWESQGSLTVTPTSLAFFQMFDSGVQAGTYASSSSTFSSLTSAIQSLADGFVAIHAEYTPSDGSLSEQFSRSNGSPTSAADLTWSYAAALTGFAARNGTQVASWGAAGLTVPATCQGSPGPTVSVTFNVDATTVWGENIYITGSVDALENWSTTTALLLSSANYPIWSITVSLPANTNIQYKYIRIDNGAVTWESDPNNSLTTPASGSYTVNDTWR

SEQ ID NO: 19 - protein, 621 aa, Sarocladium zeae
RPGPAKVQLSTRAVGDFINSETPIALEQLLCNIGANGCNSAGVSSGLVIASPSKQDPDYWYTWTRDSALVFKSIVDRFTNSYDAGLQRHITDYIVAQARLQGVSNPSGGFSDGSGLAEPKYNVDGSAFTGAWGRPQRDGPALRAIAIMSYGEWLLDNSYTDTAKNIVWPVVRNDLEYVAQYWNQTGFDLWEEVRGSSFFTIASQHQALVQGYRFAARVGASGAHYQATAPSVLCFLQSFWNPSKGYIDSNINVNDGRTGLDANSILASIHTFDASIGCDSTTFQPCSDKALSNLKAVVDSFRFYNINNGIPKGTALAVGRYAEDVYYNGNPWYLNTLAAAEQLYDAVYVWKQQGSVTVTATSRAFFADLIPNIAVGTYQSGSSTYNSIIQAVSQYGDGFVNVVATYAQSNGSLAEQFSKQDGTPLSARDLTWSYASFLTAAARRAGVIPRPWSGGVEALPGTCSAVSFTGSYTSATATNFPASQTPVTGTGTATGTSPPTTSTTAQPPSTTTACAIAPQVTVNFVARVVTNYGDTVKLVGNVDKLGNWNPGSGVVFSASDYQANNPVWKGSVVLSAGQSIQYKYVKVLSDGTVKWEADPNRTYSVPRSCATAVTRSDTWQT

SEQ ID NO: 20 - protein, 611 aa, Penicillium oxalicum
APQLSPRATASLDAWLATETTFSLNGILNNIGASGAYAKSAKNGVVIASPSTSSPNYYYTWSRDSALTLKVLIDLFRNGNLDLQTVIEEYINAQATLQTVSNPSGDLSSGAGLGEPKFNVDLSAFTDGWGRPQRDGPALRAISLIEFGNWLIDNGYSSYAINNVWPIVRNDLSYVAQYWSQTGFDLWEEVNSMSFFTVASQHRSLVEGSAFAKRVGASCSWCDSQAPQILCYMQTFWTGSYMNANTGGGRSGKDANTVLASIHTFDPEATCDDITFQPCSPRALANHKVYTDSFRSVYSINSGIAQGVAVAVGRYPEDSYYNGNPWFLSNLAAAEQLYDAIYQWNKIGSITITSTSLAFFKDIYSSAAVGTYASGSSTFTAIISAVKTYADGYVSIVQAHSYTNGSLSEQYDKSTGLSLSARDLTWSYAALLTANMRRNGVVPPSWGASSANTVPSSCSMGSAAGTYATPTATSWPSTLTSGTPGSTTSTPATSTTSTTSTSACTTPTSVAVTFDEIATTTYGENVYIVGSISQLGSWNTANAIALSASQYTSSKHLWYVTINLPAGTTFQYKYIRKESDGSIVWESDPNRSYTVPATCGTTTATENDTWR

SEQ ID NO: 21 - protein, 617 aa, Aspergillus clavatus
LTPAEWRGQSIYFLITDRFARTDGSTTAPCDLSQRAYCGGSWQGIIKQLDYIQGMGFTAIWITPITEQIPQDTAEGSAFHGYWQKDIYNVNSHFGTADDIRALSKALHDRGMYLMIDVVANHMGYNGPGASTDFSTFTPFNSASYFHSYCPINNYNDQSQVENCWLGDNTVALADLYTQHSDVRNIWYSWIKEIVGNYSADGLRIDTVKHVEKDFWTGYTQAAGVYTVGEVLDGDPAYTCPYQGYVDGVLNYPIYYPLLRAFESSSGSMGDLYNMINSVASDCKDPTVLGSFIENHDNPRFASYTKDMSQAKAVISYVILSDGIPIIYSGQEQHYSGGNDPYNREAIWLSGYSTTSELYKFIATTNKIRQLAISKDSSYLTSRNNPFYTDSNTIAMRKGSGGSQVITVLSNSGSNGGSYTLNLGNSGYSSGANLVEVYTCSSVTVGSDGKIPVPMASGLPRVLVPASWMSGSGLCGSSSTTTLVTATTTPTGSSSSTTLATAVTTPTGSCKTATTVPVVLEESVRTSYGENIFISGSIPQLGSWNPDKAVALSSSQYTSSNPLWAVTLDLPVGTSFEYKFLKKEQNGGVAWENDPNRSYTVPEACAGTSQKVDSSWR

SEQ ID NO: 22 - protein, 587 aa, Aspergillus terreus
LTPAEWRSQSIYFLLTDRFGRTDNSTTAACDTSDRVYCGGSWQGIINQLDYIQGMGFTAIWITPVTGQFYENTGDGTSYHGYWQQDIYDLNYNYGTAQDLKNLANALHERGMYLMVDVVANHMGYDGAGNTVDYSVFNPFSSSSYFHPYCLISNYDNQTNVEDCWLGDTTVSLPDLDTTSTAVRNIWYDWVADLVANYSIDGLRVDTVKHVEKDFWPGYNSAAGVYCVGEVYSGDPAYTCPYQNYMDGVLNYPIYYQLLYAFESSSGSISDLYNMISSVASSCKDPTLLGNFIENHDNPRFASYTSDYSQAKNVITFIFLSDGIPIVYAGQEQHYSGGSDPANREATWLSGYSTSATLYTWIATTNQIRSLAISKDAGYVQAKNNPFYSDSNTIAMRKGTTAGAQVITVLSNKGASGSSYTLSLSGTGYSAGATLVETYTCTTVTVDSSGNLPVPMTSGLPRVFVPSSWVNGSALCNTECTAATSISVLFEELVTTTYGENIYLSGSISQLGSWNTASAVALSASQYTSSNPEWYVSVTLPVGTSFQYKFIKKGSDGSVVWESDPNRSYTVPAGCEGATVTVADTWR

SEQ ID NO: 23 - protein, 607 aa, Aspergillus fumigatus
LTPAEWRSQSIYFLLTDRFGREDNSTTAACDVTQRLYCGGSWQGIINHLDYIQGMGFTAIWITPVTEQFYENTGDGTSYHGYWQQNIHEVNANYGTAQDLRDLANALHARGMYLMVDVVANHMGYNGAGNSVNYGVFTPFDSATYFHPYCLITDYNNQTAVEDCWLGDTTVSLPDLDTTSTAVRSIWYDWVKGLVANYSIDGLRIDTVKHVEKDFWPGYNDAAGVYCVGEVFSGDPQYTCPYQNYLDGVLNYPIYYQLLYAFQSTSGSISNLYNMISSVASDCADPTLLGNFIENHDNPRFASYTSDYSQAKNVISFMFFSDGIPIVYAGQEQHYSGGADPANREAVWLSGYSTSATLYSWIASTNKIRKLAISKDSAYITSKNNPFYYDSNTLAMRKGSVAGSQVITVLSNKGSSGSSYTLSLSGTGYSAGATLVEMYTCTTLTVDSSGNLAVPMVSGLPRVFVPSSWVSGSGLCGDSISTTATAPSATTSATATRTACAAATAIPILFEELVTTTYGESIYLTGSISQLGNWDTSSAIALSASKYTSSNPEWYVTVTLPVGTSFEYKFVKKGSDGSIAWESDPNRSYTVPTGCAGTTVTVSDTWR

SEQ ID NO: 24 - protein, 609 aa, Neosartorya fischeri
LTPAEWRSQSIYFLLTDRFGREDNSTTAACDVTQRLYCGGSWQGIINHLDYIQGMGFTAIWITPVTQQFYENTGDGTSYHGYWQQNIYEVNSNYGTAQDLRKLADALHARGMYLMVDVVANHMGYDGAGNSVDYSVFTPFDSSTYFHTYCLISDYNNQNNVEDCWLGDTTVSLPDLDTTNTAVRTIWYDWVKGLVANYSIDGLRIDTVKHVEKDFWPDYNDAAGVYCVGEVFSGDPSYTCPYQNYMDGVLNYPIYYQLLYAFQSTSGSISNLYNMISSVDSDCADPTLLGNFIENHDNPRFASYTSDYSQAKNVISFMFFSDGIPIVYAGQEQHYSGGADPANREAVWLSGYSTSATLYSWIASTNKIRKLAISKDSAYITSKNNPFYYDSNTLAMRKGSVAGSQVITVLSNKGSSGSSYTLSLSGTGYSAGATLVEMYTCTTLTVDSSGNLAVPMASGLPRVLVPSSWVSGSGLCGDSISTIATTTTSTTKTTTVATTTACASATALPILFEELVTTTYGETIYLTGSISQLGNWDTSSAIALSASKYTSSNPEWYATVTLPVGTSFQYKFFKKESDGSIVWESDPNRSYTVPAGCAGTTVTVSDTWR

SEQ ID NO: 25 - protein, 603 aa, Talaromyces emersonii
LTPAEWRKQSIYFLLTDRFGRADNSTTAACDVTERIYCGGSWQGIINHLDYIQGMGFTAIWISPVTEQLPQNTGEGEAYHGYWQQEIYTVNSNFGTSDDLLALSKALHDRGMYLMVDVVANHMGYDGDGDSVDYSVFNPFNSSSYFHPYCLITDYSNQTDVEDCWLGDTTVSLPDLNTTETVVRTIWYDWVADLVSNYSIDGLRIDTVKHVEKSFWPGYNSAAGVYCVGEVLDGDPSYTCPYQDYLDGVLNYPIYYQLLYAFESSSGSISNLYNMINSVASECSDPTLLGNFIENHDNPRFASYTSDYSLAKNVIAFIFFSDGIPIVYAGQEQHYNGGNDPYNREATWLSGYSTTAELYTFIATTNAIRSLAISVDSEYLTYKNDPFYYDSNTLAMRKGSDGLQVITVLSNLGADGSSYTLTLSGSGYSSGTELVEAYTCTTVTVDSNGDIPVPMESGLPRVFLPASSFSGSSLCSSSPSPTTTTSTSTSTTSTACTTATAVAVLFEELVTTTYGENVYLSGSISQLGDWNTDDAVALSAANYTSSNPLWYVTVTLPVGTSFEYKFIKKEENGDVEWESDPNRSYTVPTACTGATETIVDTWR

SEQ ID NO: 26 - protein, 612 aa, Penicillium funiculosum
LSAAEWRSQSIYFLLTDRFARTDGSTSAACDLSQRIQAYCGGSWQGIIDHLDYIQGMGFTAVWITPITKQMPQTTSEGTGFHGYWQQDIYSVNPNFGTADDIKALSKAIHDRGMYLMIDVVANHMGYNGAGSSTDFSVFNPFNSASYFHSYCSISDYNNQNQVENCWLGDDTVSLTDLNTQSDQVRTIWYSWVKDLVANYTVDGLRIDTVKHVEKDFWTGYSQAAGVYTLGEVLHGDPAYTCPYQGYVDGVFNYPIYYPLLNAFKSSSGSISSLVSMINSVSSDCKDPTLLGSFIENHDNPRFPSYTSDMSQAKSVIGYVFFADGIPTIYSGQEQHYAGANDPYNREAIWLSGYATDSELYKFIATANEIRKLAISKDSSYLTTRNNAFYTDSNTIAMRKGTSGSQVITVLSNSGSSGGSYTLNLNNHGYSSGAQLVELYTCASVQVDSSGNMPVPMASGLPRVLVPGSWATGSGLCGTSSGTPSKTTTLITTTSQVSSSTSSTCVAATSLPIAFTEKVTTSYGESVFITGSISQLGNWNAANAVALSASQYTSANPVWTVSLDLPVGTTFQYKYIKKEQDGSVVWESDPNRSYTVSSGCTGVKQAVSDSWR

SEQ ID NO: 27 - protein, 590 aa, Penicillium funiculosum
LTADEWRSQSIYFLLTDRFGLTSNSTTASCDVADGLYCGGSWQGVINHLDYIQGMGFTAIWITPVTENFEGDTSDGEAYHGYWQQNAYATNSHYGASSDLLKLSEALHARGMYLMVDIVVNNMAYDGAGTSVDYSIFNPFPSESYYHSYCLINYNTYNATDWDDCWEGDTIVSLPDLDTTQTYVKDTWNTWVKSFVANYSIDGLRIDSALHIQQDFFTAFEEAAGVYCIGELDYGDPAVVCPYQSVLSGVLNYPIYWQLLYAFESSSGSISNLYNMINTVKSDCADTSLLGNFIENHDNPRFAYYTSDYSEAKNVISFIFLTDGIPILYYGQEQHYSGGNIPLNREPLWTSDYSTDAQLYTYTKTSNAIRSLAIAKDSAYLTYQNYPIYQDSNTIAMRKGTTGLQLVTVLSNLGANGSSYTLTLSGSGYTSGTVVTELYTCTNVTVSSSGTIAVPMASGSPRAFLPWSSVSGSSLCNSVSSGCTAASTVAVTFEEVVTTTYGQEVYLTGSISQLGSWSTSSAVLLSAAQYTSSDPVWTVTVNLPAGESFEYKFIIVNSDGTVTWESDPNRSYTVPTGCQGLTATVDDTWR

SEQ ID NO: 28 - protein, 603 aa, Aureobasidium pullulans
LTPAQWRSQSIYQVLTDRFARTDGSTTASCDVNKYCGGSFQGIIKKLDYIQQMGFTAIWISPVVKNIYSSGQDGDSYHGYWAQDIYQVNTNFGSAADLVSLSKALHDRGMYLMVDIVTNHMGYNGCGNCVDYSIYNPFNSQSYYHPFCLINYNDQTSVEQCWAGDNTVSLPDLRTEDSNVLSMWNTWIKQLVFNYTIDGLRIDSAKSVDKAFYQPFQQAASVYAVGEVYDGDPNYFCDYQNYLDGMLNYPTYYWITQAFQSTSGSISNLYNGINTMKSTCKDTTLLGSFMENHDVARFASLTSDYALAKNAIAFTMLADGIPIIYQGQEQHFSGSSVPNNREALWLSGYPTSSQLYPFIATVNKIRKQAIKQDTGYLTYKAYPVYSDASTIVMRKGTTGSQVIGVFTNKGSSGSSSFTLSSSASGFTAGQAVTDVLSCTSYTADSNGNIAININAGAPRVLYPTSKLTGSGLCSGSSSTSGTPTTIKTSAVSGGCSTPTAVAVTFTDKVTTQYGQTIKLAGSIPQLGSWNAANAVTLSSAGYTASNPVWSGTVNIPAGQAFSYKFIKVNSDGSVTWESDPNHSYTVPASCGVTTASVSNTWQG

SEQ ID NO: 29 - protein, 624 aa, Lipomyces starkeyi
YILRRDCTTVTVLSSPESVTSSNHVQLASHEMCDSTLSASLYIYNDDYDKIVTLYYLTSSGTTGSVTASYSSSLSNNWELWSLSAPAADAVEITGASYVDSDASATYATSFDIPLTTTTTSSSSASATSTSSLTTTSSVSISVSVPTGTAANWRGRAIYQIVTDRFARTDGSTTYLCDVTDRVYCGGSYQGIINMLDYIQGMGFTAIWISPIVENIPDDTGYGYAYHGYWMKDIFALNTNFGTADDLIALATELHNRGMYLMVDIVVNHFAFSGSHADVDYSEYFPYSSQDYFHSFCWITDYSNQTNVEQCWLGDDTVPLVDVNTQLDTVKSEYQSWVQELIANYSIDGLRIDTVKHVQMDFWAPFQEAAGIYAVGEVFDGDPSYTCPYQENLDGVLNYPVYYPVVSAFESVSGSVSSLVDMIDTLKSECTDTTLLGSFLENQDNPRFPSYTSDESLIKNAIAFTMLSDGIPIIYYGQEQGLNGGNDPYNREALWLTGYSTTSTFYKYIASLNQIRNQAIYKDDTYLTYQNWVIYSDSTTIAMRKGFTGNQIITVLSNLGTSGSSYTLTLSNTGYTASSVVYEILTCTAVTVDSSGNLAVPMSSGLPKVFYQESQLVGSGICSM

SEQ ID NO: 30 - protein, 422 aa, Oryza sativa
DKILFQGFNWESWRQSGGWYNLLMGKVDDIVAAGVTHVWLPPPSHSVSTQGYMPGRLYDLDASRYGTSMELKSLISALHGKGIQAIADVVINHRCADYKDSRGIYCIFEGGTPDGRLDWGPHMICRDDTQFSDGTGNLDTGADFAAAPDIDHLNGVVQRELTDWLLWLKSDEVGFDAWRLDFARGYSPEVAKVYIEGTTPVGLAVAELWDSMAYGGDGKPEYNQDAHRQALVDWVDRVGGTASAGMVFDFTTKGIMNTAVEGELWRLIDQQGKAPGVIGWWPAKAVTFVDNHDTGSTQQMWPFPSDKVMQGYAYILTHPGNPCIFYDHFFDWGLKEQIAALVAVRQRNGVTATSSLKIMLHDADAYVAEIDGKVVMKIGSRYDVSSLIPPGFHLAAHGNGYAVWEKSAAAAADHRTSSSASL

SEQ ID NO: 31 - protein, 615 aa, Aspergillus aculeatus
AEWRTQSIYFLLTDRFGRTDNSTTATCNTGDQVYCGGTWQGIINHLDYIQGMGFTAVWISPVTEQLSANTADGESYHGYWQQKIYSLNSNFGTADDLKALSAALHERDMYLMVDVVPNHMGYAGSGDSVDYSVFDAFDSSSYFHSYCLITDWDDIDQVRTCWEGDTIVSLPDLYTTQSDVRTIWYDWIEQLVANYSIDGLRIDSALEVEPDFFTGYVSAAGVYSVGEIFNGDPATACPYQGYLDGVLNYPIYFQLLYAFESSSGSISDLYNMINSVASDCSDPTLLGNFIENHDNARFAYYTSDYSQAKNVLSFLFLSDGIPIVYAGEEQHYSGSGVPYNREATWLSGYSTTAELYQWIATTNAIRKLAISLDSNYITYKNNPFYTDSNTIAMRKGSDNLQVITILSNRGSSSSSYTLTLTGTGYAAGTTLIEAYTCTTLTVSSSGSIAVPMASGLPRVYLPASSVNKGSLCGGGTSATTATTTTTLKTTTTTTSTKTTTTSCTATTTSLPITFIELVTTTYGEEIYLTGSIAALGNWATTASGRIALSAANYSASYPEWSATVSVPVGTSFEYKFFKVGTDGSTITWESDPNRVYTVTATACAGATATVVDSWR

SEQ ID NO: 32 - protein, 590 aa, Talaromyces leycettanus
LAPAEWRKQSIYFLLTDRFGRTDNSTTATCNVSDRVYCGGSWQGIINHLDYIQGMGFTAVWISPVTEQLPQDTGDGAAYHGYWQQRIYELNANFGTESDLKALATALHDRGMYLMLDVVANHMGYAGAGNTVDYSVFDPFDSSSYFHPYCLISDYSNQTNVEDCWLGDTTVSLPDLNTTETAVQNIWYNWVAGLVANYSVDGLRIDTVKHVQKPFWPGYNKAAGVYCVGEVLNGDPSYTCDYQNYLDAVLNYPIYFQLLYAFESSSGSIANLYNMINSVASVCVDPTLLGNFIENHDNPRFAYYTSDYSQAKNVIAYIFLADGIPIVYAGQEQHYSGGNDPYNREATWLSGYSTSAELYTFIATTNQIRKLAISRDSNYLTSRNNPFYYDSNTLAMRKGSSGSQVITVLSNLGSSGSSYTLTLSNTGYSSGTSLTELHTCTSVTVDSSGNIAVPMASGSPRVLVPSSWINGSGLCSGSGTTGCTAATSVPVLFEETVTTTYGENIFISGSISQLGDWDTSQAVALSASQYTASDPLWEVTIDLPVGTSFEYKFIKVEPSGTVVWESDPNRQYTVPTACTGTTETVVATWR

SEQ ID NO: 33 - protein, 464 aa, Thermoascus aurantiacus
ATPAQWRSRSVYFLLTDRFARSDGSTTAACDTSARLDYIQGMGFTAIWISPVTEQLPQDTGDGTAYHGYWQQDIYSLNPNFGTADDLRALADALHARGMYLMVDVVANHMGYAGPGNSVDYSVFNPFNKQEYFHPYCEITNYDDQSNVEDCWLGDTIVSLPDLNTTRSDVEDIWYSWVRALVSNYSVDGLRIDTVKHVQKDFWPGYNDAAGVYCVGEVFDGDPSYTCDYQNYLDGVLNYPMYYPLLRAFSSTSGSISDLYNMINTVKAQCADSTLLGTFVENHDVPRFASYTSDIALAKNAIAFTILSDGIPIIYAGQEQHYSGGNDPANREAVWLSGYSTTSELYQFIAVSNQIRNYAIYVDEGYLTYKAWPIYQDSHTLAIRKGFDGNQVITVLSNLGSSGSSYTLSLSGTGYAAGQQVTEIYSCTDVTADSNGNIAVSMGGGLPKAFFPTAKLAGSGICWK

SEQ ID NO: 34 - protein, 633 aa, Brevibacterium halotolerans
GPAAANAETQNTSNELTAPSIKSGTILHAWNWSFNTLKHNMKDIHDAGYTAIQTSPINQVKEGNQGNKSMSNWYWLYQPTSYQIGNRYLGTEQEFKEMCAAAEEYGVKVIVDAVINHTTSDYAAISNEIKSIPNWTHGNTQIKNWSDRWDVTQNSLLGLYDWNTQNTQVQSYLKRFLERALNDGADGFRYDAAKHIELPDDGNYGSQFWPNITNTSAEFQYGEILQDSASRDAAYANYMNVTASNYGHSIRSALKNRNLSVSNISHYASEVSADKLVTWVESHDTYANDEEESTWMSDDDIRLGWAVIASRSGSTPLFFSRPEGGGNGVRFPGKSQIGDRGSALFEDQAITAVNRFHNVMDGQPEELSNPNGNNQIFMNQRGSHGVVLANAGSSSVTINTSTKLPDGRYDNKAGNGSFQVTDGKLTGTINARSVAVLYSDDIANAPHVFLENVKTGVTHSFNDQLTITLRADANTTKAVYQINNGQETVFKDGDQLTIGKGDPFGTTYTITLTGTNSDGVTRTQEYSFVKREPSAAKTIGYQNPNHWGQVNAYIYKHDGGRALELTGSWPGKAMIKNADGIYTLTLPADTDTTNAKVIFNNGSAQVPGQNQPGFDYVQNGLYNDSGLSGSLPH

(19) United States
(12) Patent Application Publication
(10) Pub. No.: US 2023/0340443 A1
(43) Pub. Date: Oct. 26, 2023

(54) GRANULAR STARCH CONVERSION ENZYMES AND METHODS

(71) Applicant: Danisco US Inc., Palo Alto, CA (US)

(72) Inventors: Bart C. Koops, Palo Alto, CA (US); Paula Johanna Maria Teunissen, Palo Alto, CA (US); Marco Van Brussel-Zwijnen, Palo Alto, CA (US); Martijn Scheffers, Palo Alto, CA (US); Kees-Jan Guijt, Palo Alto, CA (US); Zhengzheng Zou, Palo Alto, CA (US); Zhongmei Tang, Palo Alto, CA (US); Zhen Qian, Palo Alto, CA (US); Jing Ge, Palo Alto, CA (US); Zhenghong Zhang, Palo Alto, CA (US)

(21) Appl. No.: 18/151,122

(22) Filed: Jan. 6, 2023

Related U.S. Application Data
(63) Continuation of application No. 16/064,518, filed on Jun. 21, 2018, now abandoned.

(30) Foreign Application Priority Data
Dec. 21, 2015 (WO) PCT/CN2015/098121

(51) Int. Cl. C12N 9/30; C12P 19/02; C12P 19/14; C12N 9/34; C12P 7/06

(52) U.S. Cl. CPC C12N 9/242; C12P 19/02; C12P 19/14; C12Y 302/01001; C12Y 302/01003; C12N 9/2428; C12P 7/06

(57) ABSTRACT

Described are methods and compositions relating to granular starch-converting glucoamylases and α-amylases. The enzymes can be used to perform enzymatic starch hydrolysis of granular starch at or below the gelatinization temperature of insoluble granular starch.

### CROSS-REFERENCE TO RELATED APPLICATIONS

This application is a continuation of U.S. patent application Ser. No. 16/064,518, filed Jun. 21, 2018, which is a U.S. National Stage Application under 35 U.S.C. § 371 of International Patent Application No. PCT/US2016/067685, filed Dec. 20, 2016, which claims the benefit of International Application No. PCT/CN2015/098121, filed Dec. 21, 2015, which is hereby incorporated by reference in their entirety.

### REFERENCE TO A SEQUENCE LISTING

The contents of the electronic submission of the text file Sequence Listing, named “NB40781USCNT2_SequenceListing.xml” was created on May 26, 2023 and is 52 KB in size, which is hereby incorporated by reference in its entirety.

### TECHNICAL FIELD

The present methods and compositions relate to granular starch-converting glucoamylases and α-amylases. The enzymes can be used to perform enzymatic starch hydrolysis of granular starch at or below the gelatinization temperature of insoluble granular starch.

### BACKGROUND

The conversion of insoluble granular starch to glucose or other soluble saccharides like-dextrins is often part of important large-scale processes to obtain end-products, such as sugar sweeteners, specialty syrups, enzymes, proteins, alcohol (e.g., ethanol, butanol), organic acids (lactic acid, succinic acid, citric acid) and specialty biochemicals such as amino acids, (lysine, monosodium glutamate) and 1-3 propanediol. The partial crystalline nature of starch granules imparts insolubility in cold water. Solubilization of starch granules in water requires a tremendous amount of heat energy to disrupt the crystalline structure. The more water used to solubilize the granules, the more energy is required to heat the water. More energy is also required if evaporation of water from the end-product is required.

Solubilization of starch in a starch-water mixture can be performed by direct or indirect heating systems, such as direct heating by steam injection (see, for example, Starch Chemistry and Technology, eds R. L. Whistler et al., 2^{nd} Ed., 1984 Academic Press Inc., Orlando, FL and Starch Conversion Technology, Eds. G.M.A. Van Beynum et al., Food Science and Technology Series, Marcel Dekker Inc., NY). A typical conventional starch liquefaction system delivers an aqueous starch slurry under high pressure to a direct steam injection cooker that raises the slurry temperature from about 35-40° C. to 107-110° C. The slurry generally contains a thermal-stable alpha amylase in which case the pH is adjusted to favor the alpha amylase. Granular starch slurry resulting from wet milling usually has a dry solid content of 40 to 42%. The concentration is generally diluted to 32% to 35% dry solids before heating above the gelatinization temperature. Without this dilution the viscosity during the high temperature jet-cooking process would be likely so high that unit operation system cannot handle the slurry.

An alternative to the above conventional process has been described in which problems of excessive viscosity are avoided by not heating the granular starch slurry above the gelatinization temperature (see, e.g., U.S. Pat. No. 7,618,795 and US 20050136525). Instead, the granular starch is solubilized by enzymatic hydrolysis below the gelatinization temperature. Such “low-temperature” systems (known also as “no-cook” or “cold-cook”) have been reported to be able to process higher concentrations of dry solids than conventional systems (e.g., up to 45%). However, no-cook systems have the disadvantage that a relatively long incubation of about 24 hours or more at moderately elevated temperature is required for substantially complete solubilization. The longer incubation is itself associated with high energy costs.

Because of the large scale on which granular starch is processed, even seemingly small improvements in efficiency can have great economic advantage. However, the conversion process has already been extensively analyzed to identify and implement such improvements (see, e.g., Martin & Brumm at pp. 45-77 in “Starch Hydrolysis Products: Worldwide Technology, production and applications New York, VCH Publishers, Inc. 1992 and Luenser, Dev. in Ind. Microbio1.24.79-96 (1993)).

### SUMMARY

The present methods and compositions relate to granular starch-converting glucoamylases and α-amylases. The enzymes can be used to perform enzymatic starch hydrolysis of granular starch at or below the gelatinization temperature of insoluble granular starch:

1. In one aspect, a method for processing granular starch is provided, comprising: contacting a slurry comprising granular starch with a glucoamylase and a granular starch-converting α-amylase, at a temperature at or below the gelatinization temperature of the granular starch, to produce saccharides fermentable by a fermenting organism; wherein the granular starch-converting α-amylase comprises an amino acid sequence having at least 85% amino acid sequence identity to any one of SEQ ID NOs: 21-34, or at least 85% amino acid sequence identity to an active fragment, thereof.
2. In some embodiments of the method of paragraph 1, contacting the slurry with the glucoamylase and the granular starch-converting α-amylase results in increased starch conversion compared to contacting the same slurry with the same glucoamylase and α-amylase from Aspergillus kawachii (AkAA) having the amino acid sequence of SEQ ID NO: 2.
3. In some embodiments of the method of paragraph 1 or 2, contacting the slurry with the glucoamylase and the granular starch-converting α-amylase results in increased glucose release compared to contacting the same slurry with the same glucoamylase and α-amylase from Aspergillus kawachii (AkAA) having the amino acid sequence of SEQ ID NO: 2.
4. In some embodiments of the method of any of the preceding paragraphs, contacting the slurry with the glucoamylase and the granular starch-converting α-amylase results in increased total glucose equivalents compared to contacting the same slurry with the same glucoamylase and α-amylase from Aspergillus kawachii (AkAA) having the amino acid sequence of SEQ ID NO: 2.
5. In some embodiments of the method of paragraph 4, the increased total glucose equivalents is at least 5% higher, and preferably at least 10% higher, compared to the amount produced by contacting the same slurry with the glucoamylase and α-amylase from Aspergillus kawachii (AkAA) having the amino acid sequence of SEQ ID NO: 2.
6. In some embodiments of the method of any of the preceding paragraphs, the method results in the production of glucose, maltose, oligosaccharides, or a mixture thereof, optionally in the form of a syrup.
7. In some embodiments, the method of any of the preceding paragraphs further comprising contacting the saccharides with a fermenting organism to produce an end of fermentation product; wherein the contacting results in increased production of an end of fermentation product compared to contacting the same slurry with the glucoamylase and α-amylase from Aspergillus kawachii (AkAA) having the amino acid sequence of SEQ ID NO: 2.
8. In some embodiments of the method of paragraph 7, the end of fermentation product is ethanol.
9. In some embodiments of the method of paragraph 7, the end of fermentation product is a non-ethanol biochemical.
10. In some embodiments of the method of any of paragraphs 1-9, the glucoamylase and the granular starch-converting α-amylase are added simultaneously.
11. In some embodiments of the method of any of paragraphs 7-9, the glucoamylase and/or the granular starch-converting α-amylase and the fermenting organism are added simultaneously.
12. In some embodiments of the method of any of paragraphs 1-11, the glucoamylase and/or the granular starch-converting α-amylase are produced by a fermenting organism.
13. In some embodiments, the method of any of the preceding paragraphs further comprising the addition of an additional enzyme to the slurry.
14. In some embodiments of the method of any of the preceding paragraphs, the glucoamylase has at least 85% amino acid sequence identity to a glucoamylase selected from the group consisting of SEQ ID NOs: 1 and 3-20, or to an active fragment, thereof.
15. In some embodiments of the method of any of the preceding paragraphs, the glucoamylase has at least 85% amino acid sequence identity to a glucoamylase selected from the group consisting of SEQ ID NO: 1, 3, 4, 5, 7, 8, 12, 13, 16, 17, 18, 19, and 20, or to an active fragment, thereof.
16. In another aspect, a granular starch-converting α-amylase is provided, comprising an amino acid sequence having at least 85% amino acid sequence identity to any one of SEQ ID NOs: 21-34, or at least 85% amino acid sequence identity to an active fragment, thereof; wherein the granular starch-converting α-amylase, upon contacting a slurry of granular starch in combination with a glucoamylase, is capable of increased starch conversion, increased glucose release, and/or the production of increased total glucose equivalents, compared to contacting the same slurry with the same glucoamylase and α-amylase from Aspergillus kawachii (AkAA) having the amino acid sequence of SEQ ID NO: 2.
17. In some embodiments of the starch-converting α-amylase of paragraph 16; the granular starch-converting α-amylase, upon contacting a slurry of granular starch in combination with an glucoamylase, is capable of at least 5% higher, and preferably at least 10% higher, production of increased total glucose equivalents compared to contacting the same slurry with the same glucoamylase and α-amylase from Aspergillus kawachii (AkAA) having the amino acid sequence of SEQ ID NO: 2.
18. In some embodiments of the granular starch-converting α-amylase of paragraph 16 or 17; the granular starch-converting α-amylase, upon contacting a slurry of granular starch in combination with an glucoamylase and a fermenting organism, is capable of increased production of an end of fermentation product compared to contacting the same slurry with the same glucoamylase and α-amylase from Aspergillus kawachii (AkAA) having the amino acid sequence of SEQ ID NO: 2.
19. In another aspect, a composition comprising the granular starch-converting α-amylase of any of paragraphs 16-18 in combination with a glucoamylase is provided.
20. In some embodiments of the composition of paragraph 19, the glucoamylase has at least 85% amino acid sequence identity to an α-amylase selected from the group consisting of SEQ ID NOs: 1 and 3-20, or to an active fragment, thereof.
21. In some embodiments of the composition of paragraph 19 or 20, the glucoamylase has at least 85% amino acid sequence identity to an α-amylase selected from the group consisting of SEQ ID NOs: 1, 3, 4, 5, 7, 8, 12, 13, 16, 17, 18, 19, and 20, or to an active fragment, thereof.
22. In another aspect, a fermenting organism capable of producing the granular starch-converting α-amylase of any of paragraphs 16-18 optionally in combination with an glucoamylase, which glucoamylase may optionally be selected from paragraphs 20 or 22 is provided.

These and other aspects and embodiments of the compositions and methods will be apparent from the present description.

### DETAILED DESCRIPTION

### Definitions

Prior to describing the compositions and methods in detail, the following terms and abbreviations are defined.

Unless otherwise defined, all technical and scientific terms used have their ordinary meaning in the relevant scientific field. Singleton, et al., Dictionary of Microbiology and Molecular Biology, 2d Ed., John Wiley and Sons, New York (1994), and Hale & Markham, Harper Collins Dictionary of Biology, Harper Perennial, NY (1991) provide the ordinary meaning of many of the terms describing the invention.

“Starch” refers a polysaccharide composed of glucose units that occurs widely in plant tissues in the form of storage granules, consisting of amylose and amylopectin. with the formula (C6H1005)x, with X being any number. In particular, the term refers to any plant-based material, such as for example, grains, cereals, grasses, tubers and roots and more specifically wheat, barley, corn, rye, rice, sorghum, legumes, cassava, millet, potato, sweet potato, and tapioca.

“Granular starch” refers to uncooked (raw) starch, which has not been subject to gelatinization.

The term “granular starch-converting glucoamylase” refers to a glucoamylase that has increased activity on granular starch compared to the glucoamylase from Trichoderma reesei (TrGA) having the amino acid sequence of SEQ ID NO: 1, using the assays described in the Examples.

The term “granular starch-converting α-amylase” refers to an α-amylase that has increased activity on granular starch compared to the α-amylase from Aspergillus kawachii (AkAA) having the amino acid sequence of SEQ ID NO: 2, using the assays described in the Examples.

The terms “same glucoamylase” and “same α-amylase” with reference to an enzyme used for comparison purposes, refer to the identical enzyme (based on amino acid sequence) at the equivalent concentration and specific activity, such that the effect of other changes in the conditions can be experimentally evaluated.

“Starch gelatinization” means solubilization of starch molecules to form a viscous suspension.

“Gelatinization temperature” is the lowest temperature at which gelatinization of a starch containing substrate begins. The exact temperature of gelatinization depends on the specific starch and may vary depending on factors such as plant species and environmental and growth conditions. The initial starch gelatinization temperature ranges for a number of granular starches which may be used in accordance with the processes herein include barley (52-59° C.), wheat (58-64° C.), rye (57-70° C.), corn (62-72° C.), high amylose corn (67-80° C.), rice (68-77° C.), sorghum (68-77° C.), potato (58-68° C.), tapioca (59-69° C.) and sweet potato (58-72° C.) (Swinkels, pg. 32-38 in STARCH CONVERSION TECHNOLOGY, Eds Van Beynum et al., (1985) Marcel Dekker Inc. New York and The Alcohol Textbook 3.sup.rd ED. A Reference for the Beverage, Fuel and Industrial Alcohol Industries, Eds Jacques et al., (1999) Nottingham University Press, UK). Gelatinization involves melting of crystalline areas, hydration of molecules and irreversible swelling of granules. The gelatinization temperature occurs in a range for a given grain because crystalline regions vary in size and/or degree of molecular order or crystalline perfection. STARCH HYDROLYSIS PRODUCTS Worldwide Technology, Production, and Applications (eds/Shenck and Hebeda, VCH Publishers, Inc, New York, 1992) at p. 26.

“DE” or “dextrose equivalent” is an industry standard for the concentration of total reducing sugars, and is expressed as % D-glucose on a dry weight basis. Unhydrolyzed granular starch has a DE that is essentially 0 and D-glucose has a DE of 100.

“Glucose syrup” refers to an aqueous composition containing glucose solids. Glucose syrup has a DE of more than 20. Some glucose syrup contain no more than 21% water and no less than 25% reducing sugar calculated as dextrose. Some glucose syrups include at least 90% D-glucose or at least 95% D-glucose. Sometimes the terms glucose and glucose syrup are used interchangeably.

“Hydrolysis of starch” is the cleavage of glucosidic bonds in starch with the addition of water molecules.

A “slurry” is an aqueous mixture containing insoluble starch granules in water.

The term “total sugar content” refers to the total soluble sugar content present in a starch composition including monosaccharides, oligosaccharides and polysaccharides.

The term “dry solids” (ds) refer to dry solids dissolved in water, dry solids dispersed in water or a combination of both. Dry solids thus include granular starch, and its hydrolysis products, including glucose.

“Dry solid content” refers to the percentage of dry solids both dissolved and dispersed as a percentage by weight with respect to the water in which the dry solids are dispersed and/or dissolved. The initial dry solid content of starch is the weight of granular starch corrected for moisture content over the weight of granular starch plus weight of water. Subsequent dry solid content can be determined from the initial content adjusted for any water added or lost and for chemical gain. Subsequent dissolved dry solid content can be measured from refractive index as indicated below.

The term “high DS” refers to aqueous starch slurry with a dry solid content greater than 38% (wt/wt).

“Dry substance starch” refers to the dry starch content of a substrate, such as a starch slurry, and can be determined by subtracting from the mass of the substrate any contribution of non-starch components such as protein, fiber, and water. For example, if a granular starch slurry has a water content of 20% (wt/wt)., and a protein content of 1% (wt/wt), then 100 kg of granular starch has a dry starch content of 79 kg. Dry substance starch can be used in determining how many units of enzymes to use.

“Refractive Index Dry Substance” (RIDS) is the determination of the refractive index of a starch solution at a known DE at a controlled temperature then converting the RI to dry substance using an appropriate relationship, such as the Critical Data Tables of the Corn Refiners Association

“Degree of polymerization (DP)” refers to the number (n) of anhydroglucopyranose units in a given saccharide. Examples of DP1 are the monosaccharides, such as glucose and fructose. Examples of DP2 are the disaccharides, such as maltose and sucrose. A DP4+(>DP3) denotes polymers with a degree of polymerization of greater than 3.

The term “contacting” refers to the placing of referenced components (including but not limited to enzymes, substrates, and fermenting organisms) in sufficiently close proximity to affect an expect result, such as the enzyme acting on the substrate or the fermenting organism fermenting a substrate. Those skilled in the art will recognize that mixing solutions can bring about “contacting.”

The term “fermenting organism” refers to any organism, including bacterial and fungal (including filamentous fungi and yeast), suitable for producing a desired end of fermentation (EOF) product.

The term “end of fermentation (EOF) product,” or simply “fermentation product,” is any carbon-source derived molecule product that is produced by a fermenting organism, i.e., an organism capable of fermenting fermentable sugars and includes, but is not limited to, metabolites, such as citric acid, lactic acid, succinic acid, acetic acid, monosodium glutamate, gluconic acid, sodium gluconate, calcium gluconate, potassium gluconate, itaconic acid and other carboxylic acids, glucono delta-lactone, sodium erythorbate, glutamic acid, tryptophan, threonine, methionine, lysine and other amino acids, omega-3 fatty acid, isoprene, 1,3-propanediol, ethanol, methanol, propanol, butanol, other alcohols, and other biochemicals and biomaterials.

“Enzyme activity” refers to the action of an enzyme on its substrate.

An “α-amylase (E.C. class 3.2.1.1)” is an enzyme that catalyze the hydrolysis of alpha-1,4-glucosidic linkages. These enzymes have also been described as those catalysing the exo- or endohydrolysis of 1, 4-α-D-glucosidic linkages in polysaccharides containing 1, 4-α-linked D-glucose units. Another term used to describe these enzymes is glycogenase. Exemplary enzymes include alpha-1,4-glucan 4-glucanohydrase glucanohydrolase.

A “glucoamylase” refers to an amyloglucosidase class of enzymes (EC.3.2.1.3, glucoamylase, alpha-1, 4-D-glucan glucohydrolase) are enzymes that remove successive glucose units from the non-reducing ends of starch. The enzyme can hydrolyze both linear and branched glucosidic linkages of starch, amylose and amylopectin. The enzymes also hydrolyze alpha-1, 6 and alpha −1, 3 linkages although at much slower rates than alpha-1, 4 linkages.

“Pullulanase” also called debranching enzyme (E.C. 3.2.1.41, pullulan 6-glucanohydrolase), is capable of hydrolyzing alpha 1-6 glucosidic linkages in an amylopectin molecule.

“Yield” refers to the amount of a desired end-product/products (e.g., glucose) as a percentage by dry weight of the starting granular starch.

The phrase “simultaneous saccharification and fermentation (SSF)” refers to a process in the production of end of fermentation products in which a microbial organism, such as an ethanologenic microorganism, and at least one enzyme, such as one or more glucoamylase, are present during the same process step. SSF includes the contemporaneous hydrolysis of starch substrates (granular, liquefied, or solubilized) to saccharides, including glucose, and the fermentation of the saccharides into alcohol or other biochemical or biomaterial in the same reactor vessel.

Sequence identity can be determined by aligning sequences using algorithms, such as BESTFIT, FASTA, and TFASTA in the Wisconsin Genetics Software Package Release 7.0, Genetics Computer Group, 575 Science Dr., Madison, WI), using default gap parameters, or by inspection, and the best alignment (i.e., resulting in the highest percentage of sequence similarity over a comparison window). Percentage of sequence identity is calculated by comparing two optimally aligned sequences over the length of the shorter sequence (if lengths are unequal), determining the number of positions at which the identical residues occurs in both sequences to yield the number of matched positions, dividing the number of matched positions by the total number of matched and mismatched positions not counting gaps, and multiplying the result by 100 to yield the percentage of sequence identity. Unless otherwise specified, percent amino acid sequence identity as used herein is calculated using the CLUSTAL W algorithm with default parameters. See Thompson et al. (1994) Nucleic Acids Res. 22:4673-4680. Default parameters for the CLUSTAL W algorithm are:

Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF.

The term “comprising” and its cognates are used in their inclusive sense; that is, equivalent to the term “including” and its corresponding cognates.

Numeric ranges are inclusive of the numbers defining the range. Some preferred subranges are also listed, but in any case, reference to a range includes all subranges defined by integers included within a range.

The term “total glucose equivalent” refers to a manner to calculate starch conversion in a process, such as a fermentation process, so that the starch conversion in different processes can be compared. Comparing processes can be difficult because intermediate products and end products are formed next to side products. For example, in an ethanol fermentation process starch is converted into dextrins, which are converted into glucose and the glucose is fermented into ethanol by a yeast. The yeast is also converting glucose into glycerol as a main side product and bacteria present in the process can convert glucose while producing acetic acid and lactic acid. The glucose equivalent is a way in which all these soluble components, which can be measured by for example HPLC, are mathematically converted to glucose so they can be added up and form the glucose equivalent of all soluble components. For example, 1 mole a disaccharide like maltose, with a molar weight of 342.30 g/mol is converted into 2 moles glucose with a molar weight 180.02 g/mol. The mathematical conversion for maltose is then (2×180.02)/342.30=1.052 and each gram/liter of maltose is multiplied with this 1.052 to convert into the glucose equivalent for maltose. People skilled in the art will be able to do this for the other major components in the fermentation process. For DPn an average degree of polymerization of 10 is chosen. This way the glucose equivalents for ethanol, glycerol, acetic acid, lactic acid, Succinic acid, DP1, DP2, DP3 and DPn are calculated and added to form the total glucose equivalents for the process. Since only soluble components are measured, a process in which a similar amount of starch is converted will show a similar “total glucose equivalent” value. If more starch is dissolved, an increase in total glucose equivalent is visible.

### Granular Starch-Converting Glucoamylases and α-Amylases

Low-temperature starch hydrolysis processes, also known as “no-cook” or “cold-cook” processes, have been described (see, e.g., U.S. Pat. No. 7,618,795 and US 20050136525). In a cold cook process, granular starch is solubilized by enzymatic hydrolysis at or below the gelatinization temperature. Such low temperature processes represent an alternative to conventional starch hydrolysis with certain advantages, such as avoiding the high starch slurry viscosity created by heating granular starch above the gelatinization temperature and the high operational cost of such heating.

Because the cold-cook process does not require a jet cooker, it can be performed in ethanol production plants that were originally designed to use such feed stocks as sugar cane. This allows such production plants to utilize, for example, corn or sugar cane, depending on which is less expensive or more available at the time. Such plants may benefit from the use of a separation device to remove unfermentable corn material prior to introduction to the plant to avoid fouling equipment that was not designed to handle such material. Separation can be performed by centrifugation, filtration, or other conventional methods. The cost of installing a separation device is expected to be substantially less than installing a jet cooker

However, no-cook systems have the disadvantage that a relatively long incubation of about 24 hours or more at moderately elevated temperature is required for substantially complete solubilization. The longer incubation is itself associated with high energy costs and reduced throughput and the long incubation time at the moderately elevated temperature can lead to contamination.

The present compositions and methods are based on the observation that certain glucoamylases (GA) and α-amylases (AA) show a high degree of activity on granular starch. The observations are based on extensive empirical testing of a large number of GA and AA in raw starch hydrolysis assays using current commercial benchmarks as references. Because of the large number of enzymes tested, only GA and AA that performed better than benchmark enzymes, i.e., Trichoderma reesei glucoamylase (TrGA) (SEQ ID NO: 1) and Aspergillus kawachii α-amylase (AkAA) (SEQ ID NO: 2) are described, herein.

The amino acid sequences of TrGA and AkAA are shown, below:

(TrGA) from Trichoderma reesei
SEQ ID NO: 1
SVDDFISTETPIALNNLLCNVGPDGCRAFGTSAGAVIASP
STIDPDYYYMWTRDSALVFKNLIDRFTETYDAGLQRRIEQ
YITAQVTLQGLSNPSGSLADGSGLGEPKFELTLKPFTGNW
GRPQRDGPALRAIALIGYSKWLINNNYQSTVSNVIWPIVR
NDLNYVAQYWNQTGFDLWEEVNGSSFFTVANQHRALVEGA
TLAATLGQSGSAYSSVAPQVLCFLQRFWVSSGGYVDSNIN
TNEGRTGKDVNSVLTSIHTFDPNLGCDAGTFQPCSDKALS
NLKVVVDSFRSIYGVNKGIPAGAAVAIGRYAEDVYYNGNP
WYLATFAAAEQLYDAIYVWKKTGSITVTATSLAFFQELVP
GVTAGTYSSSSSTFTNIINAVSTYADGFLSEAAKYVPADG
SLAEQFDRNSGTPLSALHLTWSYASFLTATARRAGIVPPS
WANSSASTIPSTCSGASVVGSYSRPTATSFPPSQTPKPGV
PSGTPYTPLPCATPTSVAVTFHELVSTQFGQTVKVAGNAA
ALGNWSTSAAVALDAVNYADNHPLWIGTVNLEAGDVVEYK
YINVGQDGSVTWESDPNHTYTVPAVACVTQVVKEDTWQS
(AkAA) from Aspergillus kawachii
SEQ ID NO: 2
LSAAEWRTQSIYFLLTDRFGRTDNSTTATCNTGDQIYCGG
SWQGIINHLDYIQGMGFTAIWISPITEQLPQDTSDGEAYH
GYWQQKIYNVNSNFGTADDLKSLSDALHARGMYLMVDVVP
NHMGYAGNGNDVDYSVFDPFDSSSYFHPYCLITDWDNLTM
VQDCWEGDTIVSLPDLNTTETAVRTIWYDWVADLVSNYSV
DGLRIDSVEEVEPDFFPGYQEAAGVYCVGEVDNGNPALDC
PYQKYLDGVLNYPIYWQLLYAFESSSGSISNLYNMIKSVA
SDCSDPTLLGNFIENHDNPRFASYTSDYSQAKNVLSYIFL
SDGIPIVYAGEEQHYSGGDVPYNREATWLSGYDTSAELYT
WIATTNAIRKLAISADSDYITYANDPIYTDSNTIAMRKGL
PRVLLPASVVDSSSLCGGSGNTTTTTTAATSTSKATTSSS
SSSAAATTSSSCTATSTTLPITFEELVTTTYGEEVYLSGS
ISQLGEWDTSDAVKLSADDYTSSNPEWSVTVSLPVGTTFE
YKFIKVDEGGSVTWESDPNREYTVPECGSGSGETVVDTWR

The GA that performed better in combination with AkAA, or better in a blend with a different AA, are listed in the following table:

| Name | Abbr. | Source organism | SEQ ID NO |
|---|---|---|---|
| GA-1805 | AteGA1 | Aspergillus terreus | 3 |
| GA-2040 | AfuHT3 | Aspergillus fumigatus | 4 |
| GA-2331 | NfiGA1 | Neosartorya fischeri | 5 |
| GA-2437 | AfuGA2 | Neosartorya fumigata | 6 |
| GA-2439 | PmaGA1 | Penicillium marneffei | 7 |
| GA-2441 | TstGA2 | Talaromyces stipitatus | 8 |
| GA-2442 | MacGA1 | Metarhizium acridum | 9 |
| GA-2578 | ScoGA1 | Schizophyllum commune | 10 |
| GA-2722 | Tat GA2 | Trichoderma atroviridis; Hypocrea | 11 |
| | | atroviridis | |
| GA-3275 | BadGA1 | Bjerkandera adusta | 12 |
| GA-3280 | GspGA1 | Ganoderma spp | 13 |
| GA-3283 | TveGA3 | Termetes versicolor | 14 |
| GA-3294 | HsuGA3 | Hypholoma sublateritium | 15 |
| GA-3298 | FmeGA1 | Fomitiporia mediterranea | 16 |
| GA-3301 | PstGA2 | Punctularia strigosozonata | 17 |
| GA-3317 | PbrGA1 | Phlebia brevispora Nakasone | 18 |
| GA-4686 | SzeGA2 | Sarocladium zeae | 19 |
| GA-4688 | PoxGA5 | Penicillium oxalicum | 20 |

The amino acid sequences are shown, below:

GA-1805 (AteGA1) from Aspergillus terreus
SEQ ID NO: 3
APQLAPRATTSLDAWLASETTVALDGILDNVGSSGAYAKS
AKSGIVIASPSTSDPDYYYTWTRDAALTVKALIDLFRNGE
TSLQTVIMEYISSQAYLQTVSNPSGSLSTGGLAEPKYYVD
ETAYTGSWGRPQRDGPALRATAMIDFGNWLIDNGYSTYAS
SIVWPIVRNDLSYVAQYWNQTGYDLWEEVNGSSFFTIAVQ
HRALVEGSTFASKVGASCSWCDSQAPQVLCFLQRFWTGSY
IMANFGGGRSGKDANTVLGSIHTFDPNAGCDDTTFQPCSP
RALANHKVYTDSFRSIYSINSGISSGKAVAVGRYPEDSYY
NGNPWFLTTLAAAEQLYDAIYQWQKIGSITITDVSLAFFK
DLYSSAAVGTYASSSSAFTSIVSAVKTYADGYMSIVQTHA
MTNGSLSEQFGKSDGFSLSARDLTWSYAALLTANLRRNSV
VPPSWGETTATSVPSVCSATSATGTYSTATNTAWPSTLTS
GTGATTTTSKATSSSTTTTSSASSTTVECVVPTAVAVTFD
EVATTTYGENVYVVGSISQLGSWDTSKAVALSASKYTSSN
NLWYVTVTLPAGTTFQYKFIRVSSSGSVTWESDPNRSYTV
PSACGTSTAVVNTTWR;
GA-2040 (AfuHT3) from Aspergillus fumigatus
SEQ ID NO: 4
APQLSARATGSLDSWLGTETTVALNGILANIGADGAYAKS
AKPGIIIASPSTSEPDYYYTWTRDAALVTKVLVDLFRNGN
LGLQKVITEYVNSQAYLQTVSNPSGGLASGGLAEPKYNVD
MTAFTGAWGRPQRDGPALRATALIDFGNWLIDNGYSSYAV
NNIWPIVRNDLSYVSQYWSQSGFDLWEEVNSMSFFTVAVQ
HRALVEGSTFAKRVGASCSWCDSQAPQILCYMQSFWTGSY
INANTGGGRSGKDANTVLASIHTFDPEAGCDDTTFQPCSP
RALANHKVYTDSFRSVYAINSGIPQGAAVSAGRYPEDVYY
NGNPWFLTTLAAAEQLYDAIYQWKKIGSISITSTSLAFFK
DIYSSAAVGTYASSTSTFTDIINAVKTYADGYVSIVQAHA
MNNGSLSEQFDKSSGLSLSARDLTWSYAAFLTANMRRNGV
VPAPWGAASANSVPSSCSMGSATGTYSTATATSWPSTLTS
GSPGSTTTVGTTTSTTSGTAAETACATPTAVAVTFNEIAT
TTYGENVYIVGSISELGNWDTSKAVALSASKYTSSNNLWY
VSVTLPAGTTFEYKYIRKESDGSIVWESDPNRSYTVPAAC
GVSTATENDTWQ;
GA-2331 (NfiGA1) from Neosartorya fischeri
SEQ ID NO: 5
APQLSPRATGSLDSWLATESTVSLNGILANIGADGAYAKS
AKPGIIIASPSTSDPDYYYTWTRDAALVTKVLVDLFRNGN
LGLQKVITEYVNSQAYLQTVSTPSGGLSSGGLAEPKYNVD
MTAFTGAWGRPQRDGPALRATALIDFGNWLIDNGYSSYAV
NNIWPIVRNDLSYVSQYWSQSGFDLWEEVNSMSFFTVAVQ
HRALVEGSTFAKRVGASCSWCDSQAPQILCYMQSFWTGSY
INANTGGGRSGKDANTVLASIHTFDPEAGCDDTTFQPCSP
RALANHKVYTDSFRSVYAINSGIPQGVAVSAGRYPEDVYY
NGNPWFLTTLAAAEQLYDAIYQWKKIGSISITSTSLAFFK
DIYSSVAVGTYASSSSTFTAIIDAVKTYADGYVSIVEAHA
MTNGSLSEQFDKSSGMSLSARDLTWSYAALLTANMRRNGV
VPAPWGAASANSVPSSCSMGSATGTYSTATATSWPSTLTS
GSPSDTTSGTTPGTTTTTSACTTPTSVAVTFDEIATTTYG
ENVYIIGSISQLGSWDTSKAVPLSSSKYTSSNNLWYVTIN
LPAGTTFEYKYIRKESDGSIEWESDPNRSYTVPSACGVST
ATEKDTWR;
GA-2437 (AfuGA2) from Neosartorya fumigata
SEQ ID NO: 6
APQLSARATGSLDSWLGTETTVALNGILANIGADGAYAKS
AKPGIIIASPSTSEPDYYYTWTRDAALVTKVLVDLFRNGN
LGLQKVITEYVNSQAYLQTVSNPSGGLASGGLAEPKYNVD
MTAFTGAWGRPQRDGPALRATALIDFGNWLIDNGYSSYAV
NNIWPIVRNDLSYVSQYWSQSGFDLWEEVNSMSFFTVAVQ
HRALVEGSTFAKRVGASCSWCDSQAPQILCYMQSFWTGSY
INANTGGGRSGKDANTVLASIHTFDPEAGCDDTTFQPCSP
RALANHKVYTDSFRSVYAINSGIPQGAAVSAGRYPEDVYY
NGNPWFLTTLAAAEQLYDAIYQWKKIGSISITSTSLAFFK
DIYSSAAVGTYASSTSTFTDIINAVKTYADGYVSIVQAHA
MNNGSLSEQFDKSSGLSLSARDLTWSYAAFLTANMRRNGV
VPAPWGAASANSVPSSCSMGSATGTYSTATATSWPSTLTS
GSPGSTTTVGTTTSTTSGTATETACATPTAVAVTFNEIAT
TTYGENVYIVGSISELGNWDTSKAVALSASKYTSSNNLWY
VSVTLPAGTTFEYKYIRKESDGSIVWESDPNRSYTVPAAC
GVSTATENDTWR;
GA-2439 (PmaGA1) from Penicillium marneffei
SEQ ID NO: 7
APQFSPRATVGLDAWLASETTFSLNGILANIGSSGAYSAS
AKPGVVIASPSTNNPNYYYTWTRDSALTLKVLIDLFGNGN
LSLQTVIEEYINAQAYLQTVSNPSGDLSSGAGLAEPKYNV
DMSPFTGGWGRPQRDGPALRAIALIEFGNWLIDNGYSSYA
VNNIWPIVRNDLSYVSQYWSQSGFDLWEEVNSMSFFTVAN
QHRALVQGSTFAARVGASCSWCDSQAPQILCYMQTFWTGS
YINANTGGGRSGKDSNTVLTTIHTFDPEATCDDVTFQPCS
PRALANHKVYTDSFRSIYGVNSGIAQGVAVSVGRYPEDSY
YGGNPWFLSNLAAAEQLYDAIYQWNKIGSITITSTSLAFF
KDVYSSAAVGTYASGSTAFTSIISAVKTYADGYVSIVQGH
AAANGSLSEQFDRNSGVEISARDLTWSYAALLTANLRRNG
VMPPSWGAASANSVPSSCSMGSATGTYSTPTATAWPSTLT
SATGIPVTTSATASVTKATSATSTTTSATTCTTPTSVAVT
FDEIATTTYGENVFIVGSISQLGSWDTSKAIALSASQYTS
SNHLWFATLSLPAGTTFQYKYIRKESNGSIVWESDPNRSY
TVPSGCGVSTATENDTWR;
GA-2441 (TstGA2) from Talaromyces stipitatus
SEQ ID NO: 8
APGLSPRASTSLDAWLATETTVSLSGILANIGADGAYSKS
AKPGVVIASPSTDNPNYYYTWTRDSALTLKVLIDLFRNGN
LGLQTVIEEYVNAQAYLQTVSNPSGDLSSGAGLAEPKFNV
DMSAFTGSWGRPQRDGPALRAIALIDFGNWLIENGYTSLA
ANNIWPIVRNDLSYVAQYWSQSGFDLWEEVNSMSFFTVAN
QHRSLVEGSTFAAKVGASCSWCDSQAPQILCYMQTFWTGS
YMNANTGGGRSGKDANTVLTSIATFDPEATCDDVTFQPCS
PRALANHKVYTDSFRSVYGLNSGIAEGVAVAVGRYPEDSY
YNGNPWFLSNLAAAEQLYDAIYQWNKIGSITITSTSLAFF
KDVYSSAAVGTYASGSSAFTSIINAVKTYADGYISVVQSH
AMNNGSLSEQFDKNTGAELSARDLTWSYAALLTANMRRNG
VVPPSWGAASATSIPSSCTTGSAIGTYSTPTATSWPSTLT
SGTGSPGSTTSATGSVSTSVSATTTSAGSCTTPTSVAVTF
DEIATTSYGENVYIVGSISQLGSWNTANAIALSASKYTTS
NNLWYVTINLPAGTTFQYKYIRKESDGTVKWESDPNRSYT
VPSACGVSTATENDTWR;
GA-2442 (MacGA1) from Metarhizium acridum
SEQ ID NO: 9
HRDDLHGFITKQKSISLHGVLANIGSDGSRAQGAAAGAVV
ASPSKSDPDYWYTWSRDSALTFKVLIELFIGGKKSLQPKI
EQYITAQAHLQGVSNPSGGPDTGGLGEPKFHVNLTAFTGS
WGRPQRDGPPLRATALTIYANWLIANGGQAEAANTVWPII
AKDLSYTVQYWNRTGFDLWEEINGSSFFTLSASFRALVEG
ATLAKALGKQCPDCETNAPRILCFLQSFWANGYIDSNINV
NDGRTGKDVNSIISSIHTFDPAAACTDATFQPCSSRALAN
HKAVVDSFRTIYTVNKGRRPGRAAAVGRYSEDVYYNGNPW
YLATMAAAEQMYAAVYQWREIGSITVDATSLPFFSDLIPN
IAAGTYAKNSATFTSIIKAATAYGDDFVRVVKQYTPADGS
LAEQYDRETGSPKSAVHLTWSYASFVGAVERRSGIVPPSW
GEPNSNTVPKVCEAPPSCDSTMTFNVKVTTVPGESIYVVG
SITELKNWSPADAVPLDASQYTPSNPLWSAKVTIPAGTNF
EYKYIKKTSDGTVVWESDPNRSATSSTGCQSNGTLNDQWR;
GA-2578 (ScoGA1) from Schizophyllum commune
SEQ ID NO: 10
QTSAADAYVSAESPIAQAGILANIGPSGSKSHGAASGVII
ASPSTSNPDYLYTWTRDAALVSRALVDEFIEGESSLQSVI
DSYVSSQQKLQRVDNPSGSYTSGGLGEPKFNIDLTAFTGA
WGRPQRDGPALRAITLITYGNHLLSSGNTSYVTDTIWPVV
KADLDYVVSYWNQTGFDLWEEVSSSSFFTTAEQHTALRLG
ATFATAVGASASTYLTQADNVLCFLQSYWNSNGGYATANT
GGGRSGIDANTVLTSIHTFDIEAGCDSVTFQPCSDRALSN
LKVYVDSFRGLYSINPTGATDPILTGRYKEDVYYNGNPWY
LTTFAVAEQLYDALNTWDKLGSLDVTSTSLAFFKQFDSSI
TAGTYASSTSEYATLTSAIRNWADGFLEVLADFTPADGGL
TEQIDKSSGNPTSAADLTWSYASAITAFKARGGAIPASWG
AAGLTVPATCSTGGGGGSGGDTVAVTLNVQATTVYGENIY
VTGSVNQLANWSPDNAIALNADNYPTWSVTVNLPANTQIE
YKYIRKNNGQVTWESDPNRSITTSASGSFTQNDTWR;
GA-2722 Tat GA2) from Trichoderma atroviridis/
Hypocrea atroviridis
SEQ ID NO: 11
VPRLRESRHEFDIVKRSASSFLETEVPIALADLLCNIGSA
GSCAAGANSGIVIASPSKTNPDYFYTWTRDSALVFKCIVD
TFVNSYSASLQTEIENYINAQAIVQGISNPSGSLSNSGTG
LGEPKFNVDETAFTGAWGRPQRDGPALRAIALITYSKWLI
NNGYQSTANSIVWPIIQNDISYVAQYWNQTGFDLWEEVNG
SSFFTVANQHRALVEASALATSLGKSLPNASSQAAQALCF
LQSFWSSSQGYIVANINQNNGRSGKDANTLLGSIHTFDPE
GNCDASTFQPCSDRTLANHKVVVDSFRSIYTINNGIPAGT
AAAVGRYPEDSYQGGNPWYLNTLAAAELLYDALYQWKRIG
AITVTSTSLAFFKDLDSSITVGTYSSSSSTYTTLYNAVSN
YADGFVNNVATYAPSNGSLAEQYNRNNGQPLSAYDLTWSY
AALLTAAARRSGVVPYSWGETSASSVPSVCSYTSAVGSYS
SASTGSWPPNQTPTDGSGSTTSKSTSVTVSSTSTSASSTA
VATSPVTVTFDEIVTTIFGQTIKIAGNVPVLGNWNTNNAV
ALSADGYTSSNHLWNVGISFAPGTVIQYKYINVASNGDVT
WEADPNHTYTVPATGATAVTVNNSWQS;
GA-3275 (BadGA1) from Bjerkandera adusta
SEQ ID NO: 12
QSSTVDAFIASESPIARTGLLANIGADGSKASGAKSGIVI
ASPSKSNPDYFYTWTRDAALVFKAIIDRYTSGEDTATRRQ
IDEYVSGQALLQQVSNPSGTVSTGGLAEPKYNVDMSAFTG
GWGRPQRDGPALRATAIIAYANWLVANGNTSYVTSTLWPV
LKLDLDYVRDNWNQTGFDLWEEINSSSFFTTAVQHRALRE
GNALAAKIGQTVSGYTTQADNVLCFLQSYWNPSGGFATSN
TGGGRSGKDANSVLTSIHTFDAAAGCDALTFQPCSDRALS
NHKVYVDSFRSIYSVNSGIASNAAVATGRYPEDSYYGGNP
WYLTTLAAAEQLYDALTVWDAQGSLNVTSVSLAFFQQFAP
TVTAGTYPASSATYGTLTAAIRAYADGFVAVVAKYTPSNG
GLAEQYTRAGGTPTSAADLTWSYAAALTAFSAREGFTPAS
WGAKGLTAPAACNTNSGGGSGGGSGNTVAVTFNVQATTVW
GENIYLTGSVDALQNWSPDNALLLSSANYPTWSITVNLPP
STAIQYKYIRKNNGAVTWESDPNMSITTPGSGSATLSDTW
R;
GA-3280 (GspGA1) from Ganoderma spp.
SEQ ID NO: 13
QSSADAYVASEASIAKAGLLANIGANGSKSEGAKAGIVVA
SPSTSNPDYLYTWTRDSSLVFKTVIDQFTTGEDTSLRGLI
DEFTAAQSILQQTSNPSGSVSTGGLGEPKFNVDETAFTGA
WGRPQRDGPALRATAIITYANWLLANGNGTSYVQNTLWPI
IKLDLDYVENNWNQSTFDLWEEVNSSSFFTTAVQHRALRE
GVALASAIGQTSVVSGYSAQADNLLCFLQSYWNSGSGFVT
ANTGGGRSGRDANTVLTSIHTFDVEAGCDAVTFQPCSDKA
LSNLKVYVDAFRSIYGINSGIASNAAVATGRYPEDSYYNG
NPWYLAVFAVAEQLYDALITWDELGSLNVTSTSLAFFQQF
DSSVTAGTYDSSSSTYSTLTSGIKGFADGFLEVNSKYTPS
TGALSEQFDKSSGSQLSASDLTWSYAAALTAFAARSGKTY
ASWGAAGLITTCGGSGGGGGGSGTVSVTFNVQATTVFGEN
IYITGSVDALQNWSPDNALILSAANYPIWSITVSLPASTV
IEYKYIRKFNGQVTWESDPNDSITTPASGSYIENDTWR;
GA-3283 (TveGA3) Termetes versicolor
SEQ ID NO: 14
QSSVADAYVASESSIAKAGVLANIGPSGSKSQGAKAGIVV
ASPSTTNPDYLFTWTRDTSLVFKALIDQLTSGEDPSLRGL
VDMFTSSQAALQQVSNPSGTVSTGGLGEPKFNIDESAFTG
AWGRPQRDGPALRSTAFISYANWLLDNGNTTYVTQTLWPV
IKLDLDYVEANWNQTGFDLWEEVNSSSFFTTAVQHRALRE
GAAFATRIGQTSVVSGYTTQAANVLCFLQSYWNPSGGFVT
ANTGGGRSGRDANTVLTSIHTFDPAAGCDATTFQPCSDKA
LSNLKVYVDAFRSIYTINSGIAANAAVATGRYPEDSYQGG
NPWYLATIAVAEQLYDALIVWDQLGSINVTTTSLPFFQQF
SSTVTTGTFASTSATYTTLTTAVRNFADGFIAVNAQFTPS
NGGLAEQFSRSNGQPVSAVDLTWSYAATLTAFHARAGLTY
PGWGAAGLTVPAVCSTSGSGSGGGGAGTVAVTFNVQATTF
FGENIYITGSVDALQNWSPDNALLLSSANYPIWSITVNLP
ASTSVQYKFIRKAPGELIWESDPNNQITTPASGTFTQSDT
FR;
GA-3294 (HsuGA3) from Hypholoma sublateritium
SEQ ID NO: 15
QSSAVSSYLATESVIAKAGLLANIGPSGSKASGAVSGVVV
AAPSTNPDYIFTWTRDSALVFKAIIDSFARGEDATLRTSI
DQYVAAQKIQQQVSNPSGTVSSGGLGEPKFNVDLSAFTGA
WGRPQRDGPALRATALISYGNSLISASNTSYVLANIWPLV
KLDLDYVAANWNQTGFDLWEEVNSSSFFTTAVQHRALRQG
AAFATALGOTASVAGYTTQAANVLCFLQSYWNPSQGYITA
NTGGGRSGKDANTALASIHTFDPTAGCDAATFQPCSDKAL
SSLKVYVDSFRSIYTVNSAVASPGAVATGRYPEDSYFGGN
PWYLATMAVAEQLYDALIVWKAQGSLNVTSTSLAFFQQFS
SAVTVGTYASTTATFTTLTTAIANQADGFVAIVQEFTPST
GSLSEQYSRSNGAQLSANDLTWSYASILTAVTARNGLAGD
NWGAAGLVVPSSCSTSGTGSSSGGGSSGTVAVTFKVTATT
TFGENIYLTGSDDALEDWSPTSTLILSAATYPVWSITVNL
PASTALQYKYIRIFNGVTTWESDPNNAFTTVASGTQTLTD
TWR;
GA-3298 (FmeGA1) from Fomitiporia
mediterranea
SEQ ID NO: 16
QTAVDSYVATESPIAKTNLLANIGASGSKSQGAKPGIVIAS
PSTTNPNYLFTWTRDSSLVFKTIIDQYTNGQDTSLRTLID
EFVSAEATLQQVSNPSGTVSTGGLGEPKFNIDETAFTGAW
GRPQRDGPALRATAIINYANYLLANDNSSFVTNTLWPILQ
LDLDYVAQDWNQTTFDLWEEVDSTSFFTAAVQHRSLREGA
TLATKIGQTSVVSGYTTQAENILCFMQSFWNAGGNFMTAN
TGGGRSGKDANTVLASIHTFDSSAGCDAATFQPCSDRALA
NLKTYVDAFRSIYSINSGIASNAAVATGRYPEDVYFNGNP
WYLTTLSVAEQLYDAITVWNAQGSLNVTSVSQPFFALFQS
DIAVGTYASSSSTFTSLLSSIKSFADGFVSVVAKYTPSNG
GLSEQYSKSDGTPTSAVDLTWSYAAALTAFAARDGFVPAS
WGAAGLTVPSTCSTSGSGPGSGGTVAVTFNVQATTVFGEN
IYITGSVDALQNWSPDNAIILSAANYPTWSVTINLPASTT
IQYKYIRKENGA VTWESDPNMQITTPSGGTFIENDVWR;
GA-3301 (PstGA2) from Punctularia
strigosozonata
SEQ ID NO: 17
QTASAAAYATTEAPIAKAGVLANIGPSGSKSQGAKAGIVI
ASPSTSNPDYLYTWTRDSSLVFKALIDQYTSGIDTTLRGA
IDNFFNAEKILQQVSNPSGTVSTGGLGEPKFNIDETAFTG
AWGRPQRDGPALRATALITYANYLYSTGNTTFVSNTLWPV
IKLDLDYAANNWNQTTFDLWEEVSSSSFFTTAVQHRSLRE
GATLATKLGVTSSASTYTSAASSLLCFLQSYWNPAGGYIT
ANTGGGRSGKDANTVLTSIHTFDPAAGCDAVTFQPCSDKA
LSNLKVYVDSFRSIYGINSGIASNAAVATGRYPEDTYYNG
NPWYLTTLAVAEQLYDALIVWNARGSLNVTSTSLAFFQQF
SSSVTTGTYPSTSTTFTTLTSAVKTFADGFVAVVAKYTPS
SGALSEQFDKSSGSQLSAADLTWSYAAALTAFEARNGTTF
ASWGAAGLTTSCSSSGSGSGGGSGSSGSVPVNFQETATTV
YGENIFIVGSISPLGNWDPNSAIALSAANYPNWQVSISLP
ASTTFQYKYIRKYNGAVTWESDPNRSFTTPSSGSYNENDT
WR;
GA-3317 (PbrGA1) from Phlebia brevispora
Nakasone
SEQ ID NO: 18
QTNVNSYVASESAFAKAGLLANIGPSGSKSSGADPGIVIA
SPSTTNPDYLYTWVRDSSLVFKVLIDQYTTGVDTSLRTLI
DEFVSAEAILQQVTNPSGSVTTGGLGEPKFNIDETAFTGS
WGRPQRDGPALRSTAIITYANWLLDNGNTSYVTETLWPVL
ELDLNYVMNNWNQSTYDLWEEIDSSSFWTTAVQHRALRQG
SALATRIGQTSMVSGYNTQAANVLCFLQSFWNPSGNYVTA
NTGGGRSGIDANTVLTSIHTFDPSAGCDATTFQPCSDKAL
ANLKVYTDSFRSIYSVNTGIASNAAVATGRYPEDVYMGGN
PWYLATMAAAEQLYDALSVWESQGSLTVTPTSLAFFQMFD
SGVQAGTYASSSSTFSSLTSAIQSLADGFVAIHAEYTPSD
GSLSEQFSRSNGSPTSAADLTWSYAAALTGFAARNGTQVA
SWGAAGLTVPATCQGSPGPTVSVTFNVDATTVWGENIYIT
GSVDALENWSTTTALLLSSANYPIWSITVSLPANTNIQYK
YIRIDNGAVTWESDPNNSLTTPASGSYTVNDTWR;
GA-4686 (SzeGA2) from Sarocladium zeae
SEQ ID NO: 19
RPGPAKVQLSTRAVGDFINSETPIALEQLLCNIGANGCNS
AGVSSGLVIASPSKQDPDYWYTWTRDSALVFKSIVDRFTN
SYDAGLQRHITDYIVAQARLQGVSNPSGGFSDGSGLAEPK
YNVDGSAFTGAWGRPQRDGPALRAIAIMSYGEWLLDNSYT
DTAKNIVWPVVRNDLEYVAQYWNQTGFDLWEEVRGSSFFT
IASQHQALVQGYRFAARVGASGAHYQATAPSVLCFLQSFW
NPSKGYIDSNINVNDGRTGLDANSILASIHTFDASIGCDS
TTFQPCSDKALSNLKAVVDSFRFYNINNGIPKGTALAVGR
YAEDVYYNGNPWYLNTLAAAEQLYDAVYVWKQQGSVTVTA
TSRAFFADLIPNIAVGTYQSGSSTYNSIIQAVSQYGDGFV
NVVATYAQSNGSLAEQFSKQDGTPLSARDLTWSYASFLTA
AARRAGVIPRPWSGGVEALPGTCSAVSFTGSYTSATATNF
PASQTPVTGTGTATGTSPPTTSTTAQPPSTTTACAIAPQV
TVNFVARVVTNYGDTVKLVGNVDKLGNWNPGSGVVFSASD
YQANNPVWKGSVVLSAGQSIQYKYVKVLSDGTVKWEADPN
RTYSVPRSCATAVTRSDTWQT;
GA-4688 (PoxGA5) from Penicillium oxalicum
SEQ ID NO: 20
APQLSPRATASLDAWLATETTFSLNGILNNIGASGAYAKS
AKNGVVIASPSTSSPNYYYTWSRDSALTLKVLIDLFRNGN
LDLQTVIEEYINAQATLQTVSNPSGDLSSGAGLGEPKFNV
DLSAFTDGWGRPQRDGPALRAISLIEFGNWLIDNGYSSYA
INNVWPIVRNDLSYVAQYWSQTGFDLWEEVNSMSFFTVAS
QHRSLVEGSAFAKRVGASCSWCDSQAPQILCYMQTFWTGS
YMNANTGGGRSGKDANTVLASIHTFDPEATCDDITFQPCS
PRALANHKVYTDSFRSVYSINSGIAQGVAVAVGRYPEDSY
YNGNPWFLSNLAAAEQLYDAIYQWNKIGSITITSTSLAFF
KDIYSSAAVGTYASGSSTFTAIISAVKTYADGYVSIVQAH
SYTNGSLSEQYDKSTGLSLSARDLTWSYAALLTANMRRNG
VVPPSWGASSANTVPSSCSMGSAAGTYATPTATSWPSTLT
SGTPGSTTSTPATSTTSTTSTSACTTPTSVAVTFDEIATT
TYGENVYIVGSISQLGSWNTANAIALSASQYTSSKHLWYV
TINLPAGTTFQYKYIRKESDGSIVWESDPNRSYTVPATCG
TTTATENDTWR;

The AA that performed better in combination with TrGA, or better in a blend with a different GA, are listed in the following table:

| Name | Abbr. | Source organism | SEQ ID NO* |
|---|---|---|---|
| AA-1704 | AcAA | Aspergillus clavatus | 21 |
| AA-1708 | AtAA | Aspergillus terreus | 22 |
| AA-2115 | AfuAmy1 | Aspergillus fumigatus Af293 | 23 |
| AA-2205 | NfiAmy1 | Neosartorya fischeri | 24 |
| AA-2285 | TemAmy1 | Talaromyces emersonii | 25 |
| AA-2301 | PfuAmy1 | Penicillium funiculosum | 26 |
| AA-2303 | PfuAmy3 | Penicillium funiculosum | 27 |
| AA-2506 | ApuAmy1 | Aureobasidium pullulans | 28 |
| AA-2522 | LstAmy1 | Lipomyces starkeyi | 29 |
| AA-2676 | OsaAmy2 | Oryza sativa Japonica Group | 30 |
| AA-2940 | AacAmy2 | Aspergillus aculeatus | 31 |
| AA-3238 | TleAmy1 | Talaromyces leycettanus | 32 |
| AA-3239 | TauAmy1 | Thermoascus aurantiacus | 33 |
| AA-3937 | BhaAmy3 | Brevibacterium halotolerans strain | 34 |
| | | XFB-BI | |

The amino acid sequences are shown, below:

AA-1704 (AcAA) from Aspergillus clavatus
SEQ ID NO: 21
LTPAEWRGQSIYFLITDRFARTDGSTTAPCDLSQRAYCGG
SWQGIIKQLDYIQGMGFTAIWITPITEQIPQDTAEGSAFH
GYWQKDIYNVNSHFGTADDIRALSKALHDRGMYLMIDVVA
NHMGYNGPGASTDFSTFTPFNSASYFHSYCPINNYNDQSQ
VENCWLGDNTVALADLYTQHSDVRNIWYSWIKEIVGNYSA
DGLRIDTVKHVEKDFWTGYTQAAGVYTVGEVLDGDPAYTC
PYQGYVDGVLNYPIYYPLLRAFESSSGSMGDLYNMINSVA
SDCKDPTVLGSFIENHDNPRFASYTKDMSQAKAVISYVIL
SDGIPIIYSGQEQHYSGGNDPYNREAIWLSGYSTTSELYK
FIATTNKIRQLAISKDSSYLTSRNNPFYTDSNTIAMRKGS
GGSQVITVLSNSGSNGGSYTLNLGNSGYSSGANLVEVYTC
SSVTVGSDGKIPVPMASGLPRVLVPASWMSGSGLCGSSST
TTLVTATTTPTGSSSSTTLATAVTTPTGSCKTATTVPVVL
EESVRTSYGENIFISGSIPQLGSWNPDKAVALSSSQYTSS
NPLWAVTLDLPVGTSFEYKFLKKEQNGGVAWENDPNRSYT
VPEACAGTSQKVDSSWR;
AA-1708 (AtAA) from Aspergillus terreus
SEQ ID NO: 22
LTPAEWRSQSIYFLLTDRFGRTDNSTTAACDTSDRVYCGG
SWQGIINQLDYIQGMGFTAIWITPVTGQFYENTGDGTSYH
GYWQQDIYDLNYNYGTAQDLKNLANALHERGMYLMVDVVA
NHMGYDGAGNTVDYSVFNPFSSSSYFHPYCLISNYDNQTN
VEDCWLGDTTVSLPDLDTTSTAVRNIWYDWVADLVANYSI
DGLRVDTVKHVEKDFWPGYNSAAGVYCVGEVYSGDPAYTC
PYQNYMDGVLNYPIYYQLLYAFESSSGSISDLYNMISSVA
SSCKDPTLLGNFIENHDNPRFASYTSDYSQAKNVITFIFL
SDGIPIVYAGQEQHYSGGSDPANREATWLSGYSTSATLYT
WIATTNOIRSLAISKDAGYVQAKNNPFYSDSNTIAMRKGT
TAGAQVITVLSNKGASGSSYTLSLSGTGYSAGATLVETYT
CTTVTVDSSGNLPVPMTSGLPRVFVPSSWVNGSALCNTEC
TAATSISVLFEELVTTTYGENIYLSGSISQLGSWNTASAV
ALSASQYTSSNPEWYVSVTLPVGTSFQYKFIKKGSDGSVV
WESDPNRSYTVPAGCEGATVTVADTWR;
AA-2115 (AfuAmy1) from Aspergillus fumigatus
Af293
SEQ ID NO: 23
LTPAEWRSQSIYFLLTDRFGREDNSTTAACDVTQRLYCGG
SWQGIINHLDYIQGMGFTAIWITPVTEQFYENTGDGTSYH
GYWQQNIHEVNANYGTAQDLRDLANALHARGMYLMVDVVA
NHMGYNGAGNSVNYGVFTPFDSATYFHPYCLITDYNNQTA
VEDCWLGDTTVSLPDLDTTSTAVRSIWYDWVKGLVANYSI
DGLRIDTVKHVEKDFWPGYNDAAGVYCVGEVFSGDPQYTC
PYQNYLDGVLNYPIYYQLLYAFQSTSGSISNLYNMISSVA
SDCADPTLLGNFIENHDNPRFASYTSDYSQAKNVISFMFF
SDGIPIVYAGQEQHYSGGADPANREAVWLSGYSTSATLYS
WIASTNKIRKLAISKDSAYITSKNNPFYYDSNTLAMRKGS
VAGSQVITVLSNKGSSGSSYTLSLSGTGYSAGATLVEMYT
CTTLTVDSSGNLAVPMVSGLPRVFVPSSWVSGSGLCGDSI
STTATAPSATTSATATRTACAAATAIPILFEELVTTTYGE
SIYLTGSISQLGNWDTSSAIALSASKYTSSNPEWYVTVTL
PVGTSFEYKFVKKGSDGSIAWESDPNRSYTVPTGCAGTTV
TVSDTWR;
AA-2205 (NfiAmy1) from Neosartorya fischeri
SEQ ID NO: 24
LTPAEWRSQSIYFLLTDRFGREDNSTTAACDVTQRLYCGG
SWQGIINHLDYIQGMGFTAIWITPVTQQFYENTGDGTSYH
GYWQQNIYEVNSNYGTAQDLRKLADALHARGMYLMVDVVA
NHMGYDGAGNSVDYSVFTPFDSSTYFHTYCLISDYNNQNN
VEDCWLGDTTVSLPDLDTTNTAVRTIWYDWVKGLVANYSI
DGLRIDTVKHVEKDFWPDYNDAAGVYCVGEVFSGDPSYTC
PYQNYMDGVLNYPIYYQLLYAFQSTSGSISNLYNMISSVD
SDCADPTLLGNFIENHDNPRFASYTSDYSQAKNVISFMFF
SDGIPIVYAGQEQHYSGGADPANREAVWLSGYSTSATLYS
WIASTNKIRKLAISKDSAYITSKNNPFYYDSNTLAMRKGS
VAGSQVITVLSNKGSSGSSYTLSLSGTGYSAGATLVEMYT
CTTLTVDSSGNLAVPMASGLPRVLVPSSWVSGSGLCGDSI
STIATTTTSTTKTTTVATTTACASATALPILFEELVTTTY
GETIYLTGSISQLGNWDTSSAIALSASKYTSSNPEWYATV
TLPVGTSFQYKFFKKESDGSIVWESDPNRSYTVPAGCAGT
TVTVSDTWR;
AA-2285 (TemAmy1) from Talaromyces emersonii
SEQ ID NO: 25
LTPAEWRKQSIYFLLTDRFGRADNSTTAACDVTERIYCGG
SWQGIINHLDYIQGMGFTAIWISPVTEQLPQNTGEGEAYH
GYWQQEIYTVNSNFGTSDDLLALSKALHDRGMYLMVDVVA
NHMGYDGDGDSVDYSVFNPFNSSSYFHPYCLITDYSNQTD
VEDCWLGDTTVSLPDLNTTETVVRTIWYDWVADLVSNYSI
DGLRIDTVKHVEKSFWPGYNSAAGVYCVGEVLDGDPSYTC
PYQDYLDGVLNYPIYYQLLYAFESSSGSISNLYNMINSVA
SECSDPTLLGNFIENHDNPRFASYTSDYSLAKNVIAFIFF
SDGIPIVYAGQEQHYNGGNDPYNREATWLSGYSTTAELYT
FIATTNAIRSLAISVDSEYLTYKNDPFYYDSNTLAMRKGS
DGLQVITVLSNLGADGSSYTLTLSGSGYSSGTELVEAYTC
TTVTVDSNGDIPVPMESGLPRVFLPASSFSGSSLCSSSPS
PTTTTSTSTSTTSTACTTATAVAVLFEELVTTTYGENVYL
SGSISQLGDWNTDDAVALSAANYTSSNPLWYVTVTLPVGT
SFEYKFIKKEENGDVEWESDPNRSYTVPTACTGATETIVD
TWR;
AA-2301 (PfuAmy1) from Penicillium funiculosum
SEQ ID NO: 26
LSAAEWRSQSIYFLLTDRFARTDGSTSAACDLSQRIQAYC
GGSWQGIIDHLDYIQGMGFTAVWITPITKQMPQTTSEGTG
FHGYWQQDIYSVNPNFGTADDIKALSKAIHDRGMYLMIDV
VANHMGYNGAGSSTDFSVFNPFNSASYFHSYCSISDYNNQ
NQVENCWLGDDTVSLTDLNTQSDQVRTIWYSWVKDLVANY
TVDGLRIDTVKHVEKDFWTGYSQAAGVYTLGEVLHGDPAY
TCPYQGYVDGVFNYPIYYPLLNAFKSSSGSISSLVSMINS
VSSDCKDPTLLGSFIENHDNPRFPSYTSDMSQAKSVIGYV
FFADGIPTIYSGQEQHYAGANDPYNREAIWLSGYATDSEL
YKFIATANEIRKLAISKDSSYLTTRNNAFYTDSNTIAMRK
GTSGSQVITVLSNSGSSGGSYTLNLNNHGYSSGAQLVELY
TCASVQVDSSGNMPVPMASGLPRVLVPGSWATGSGLCGTS
SGTPSKTTTLITTTSQVSSSTSSTCVAATSLPIAFTEKVT
TSYGESVFITGSISQLGNWNAANAVALSASQYTSANPVWT
VSLDLPVGTTFQYKYIKKEQDGSVVWESDPNRSYTVSSGC
TGVKQAVSDSWR;
AA-2303 (PfuAmy3) from Penicillium funiculosum
SEQ ID NO: 27
LTADEWRSQSIYFLLTDRFGLTSNSTTASCDVADGLYCGG
SWQGVINHLDYIQGMGFTAIWITPVTENFEGDTSDGEAYH
GYWQQNAYATNSHYGASSDLLKLSEALHARGMYLMVDIVV
NNMAYDGAGTSVDYSIFNPFPSESYYHSYCLINYNTYNAT
DWDDCWEGDTIVSLPDLDTTQTYVKDTWNTWVKSFVANYS
IDGLRIDSALHIQQDFFTAFEEAAGVYCIGELDYGDPAVV
CPYQSVLSGVLNYPIYWQLLYAFESSSGSISNLYNMINTV
KSDCADTSLLGNFIENHDNPRFAYYTSDYSEAKNVISFIF
LTDGIPILYYGQEQHYSGGNIPLNREPLWTSDYSTDAQLY
TYTKTSNAIRSLAIAKDSAYLTYQNYPIYQDSNTIAMRKG
TTGLQLVTVLSNLGANGSSYTLTLSGSGYTSGTVVTELYT
CTNVTVSSSGTIAVPMASGSPRAFLPWSSVSGSSLCNSVS
SGCTAASTVAVTFEEVVTTTYGQEVYLTGSISQLGSWSTS
SAVLLSAAQYTSSDPVWTVTVNLPAGESFEYKFIIVNSDG
TVTWESDPNRSYTVPTGCQGLTATVDDTWR;
AA-2506 (ApuAmy1) from Aureobasidium pullulans
SEQ ID NO: 28
LTPAQWRSQSIYQVLTDRFARTDGSTTASCDVNKYCGGSF
QGIIKKLDYIQQMGFTAIWISPVVKNIYSSGQDGDSYHGY
WAQDIYQVNTNFGSAADLVSLSKALHDRGMYLMVDIVTNH
MGYNGCGNCVDYSIYNPFNSQSYYHPFCLINYNDQTSVEQ
CWAGDNTVSLPDLRTEDSNVLSMWNTWIKQLVFNYTIDGL
RIDSAKSVDKAFYQPFQQAASVYAVGEVYDGDPNYFCDYQ
NYLDGMLNYPTYYWITQAFQSTSGSISNLYNGINTMKSTC
KDTTLLGSFMENHDVARFASLTSDYALAKNAIAFTMLADG
IPIIYQGQEQHFSGSSVPNNREALWLSGYPTSSQLYPFIA
TVNKIRKQAIKQDTGYLTYKAYPVYSDASTIVMRKGTTGS
QVIGVFTNKGSSGSSSFTLSSSASGFTAGQAVTDVLSCTS
YTADSNGNIAININAGAPRVLYPTSKLTGSGLCSGSSSTS
GTPTTIKTSAVSGGCSTPTAVAVTFTDKVTTQYGQTIKLA
GSIPQLGSWNAANAVTLSSAGYTASNPVWSGTVNIPAGQA
FSYKFIKVNSDGSVTWESDPNHSYTVPASCGVTTASVSNT
WQG;
AA-2522 (LstAmy1) from Lipomyces starkeyi
SEQ ID NO: 29
YILRRDCTTVTVLSSPESVTSSNHVQLASHEMCDSTLSAS
LYIYNDDYDKIVTLYYLTSSGTTGSVTASYSSSLSNNWEL
WSLSAPAADAVEITGASYVDSDASATYATSFDIPLTTTTT
SSSSASATSTSSLTTTSSVSISVSVPTGTAANWRGRAIYQ
IVTDRFARTDGSTTYLCDVTDRVYCGGSYQGIINMLDYIQ
GMGFTAIWISPIVENIPDDTGYGYAYHGYWMKDIFALNTN
FGTADDLIALATELHNRGMYLMVDIVVNHFAFSGSHADVD
YSEYFPYSSQDYFHSFCWITDYSNQTNVEQCWLGDDTVPL
VDVNTQLDTVKSEYQSWVQELIANYSIDGLRIDTVKHVQM
DFWAPFQEAAGIYAVGEVFDGDPSYTCPYQENLDGVLNYP
VYYPVVSAFESVSGSVSSLVDMIDTLKSECTDTTLLGSFL
ENQDNPRFPSYTSDESLIKNAIAFTMLSDGIPIIYYGQEQ
GLNGGNDPYNREALWLTGYSTTSTFYKYIASLNQIRNQAI
YKDDTYLTYQNWVIYSDSTTIAMRKGFTGNQIITVLSNLG
TSGSSYTLTLSNTGYTASSVVYEILTCTAVTVDSSGNLAV
PMSSGLPKVFYQESQLVGSGICSM;
AA-2676 (OsaAmy2) from Oryza sativa Japonica
Group
SEQ ID NO: 30
DKILFQGFNWESWRQSGGWYNLLMGKVDDIVAAGVTHVWL
PPPSHSVSTQGYMPGRLYDLDASRYGTSMELKSLISALHG
KGIQAIADVVINHRCADYKDSRGIYCIFEGGTPDGRLDWG
PHMICRDDTQFSDGTGNLDTGADFAAAPDIDHLNGVVQRE
LTDWLLWLKSDEVGFDAWRLDFARGYSPEVAKVYIEGTTP
VGLAVAELWDSMAYGGDGKPEYNQDAHRQALVDWVDRVGG
TASAGMVFDFTTKGIMNTAVEGELWRLIDQQGKAPGVIGW
WPAKAVTFVDNHDTGSTQQMWPFPSDKVMQGYAYILTHPG
NPCIFYDHFFDWGLKEQIAALVAVRQRNGVTATSSLKIML
HDADAYVAEIDGKVVMKIGSRYDVSSLIPPGFHLAAHGNG
YAVWEKSAAAAADHRTSSSASL;
AA-2940 (AacAmy2) from Aspergillus aculeatus
SEQ ID NO: 31
AEWRTQSIYFLLTDRF
GRTDNSTTATCNTGDQVYCGGTWQGIINHLDYIQGMGFTA
VWISPVTEQLSANTADGESYHGYWQQKIYSLNSNFGTADD
LKALSAALHERDMYLMVDVVPNHMGYAGSGDSVDYSVFDA
FDSSSYFHSYCLITDWDDIDQVRTCWEGDTIVSLPDLYTT
QSDVRTIWYDWIEQLVANYSIDGLRIDSALEVEPDFFTGY
VSAAGVYSVGEIFNGDPATACPYQGYLDGVLNYPIYFQLL
YAFESSSGSISDLYNMINSVASDCSDPTLLGNFIENHDNA
RFAYYTSDYSQAKNVLSFLFLSDGIPIVYAGEEQHYSGSG
VPYNREATWLSGYSTTAELYQWIATTNAIRKLAISLDSNY
ITYKNNPFYTDSNTIAMRKGSDNLQVITILSNRGSSSSSY
TLTLTGTGYAAGTTLIEAYTCTTLTVSSSGSIAVPMASGL
PRVYLPASSVNKGSLCGGGTSATTATTTTTLKTTTTTTST
KTTTTSCTATTTSLPITFIELVTTTYGEEIYLTGSIAALG
NWATTASGRIALSAANYSASYPEWSATVSVPVGTSFEYKF
FKVGTDGSTITWESDPNRVYTVTATACAGATATVVDSWR;
AA-3238 (TleAmy1) Talaromyces leycettanus
SEQ ID NO: 32
LAPAEWRKQSIYFLLTDRFGRTDNSTTATCNVSDRVYCGG
SWQGIINHLDYIQGMGFTAVWISPVTEQLPQDTGDGAAYH
GYWQQRIYELNANFGTESDLKALATALHDRGMYLMLDVVA
NHMGYAGAGNTVDYSVFDPFDSSSYFHPYCLISDYSNQTN
VEDCWLGDTTVSLPDLNTTETAVQNIWYNWVAGLVANYSV
DGLRIDTVKHVQKPFWPGYNKAAGVYCVGEVLNGDPSYTC
DYQNYLDAVLNYPIYFQLLYAFESSSGSIANLYNMINSVA
SVCVDPTLLGNFIENHDNPRFAYYTSDYSQAKNVIAYIFL
ADGIPIVYAGQEQHYSGGNDPYNREATWLSGYSTSAELYT
FIATTNOIRKLAISRDSNYLTSRNNPFYYDSNTLAMRKGS
SGSQVITVLSNLGSSGSSYTLTLSNTGYSSGTSLTELHTC
TSVTVDSSGNIAVPMASGSPRVLVPSSWINGSGLCSGSGT
TGCTAATSVPVLFEETVTTTYGENIFISGSISQLGDWDTS
QAVALSASQYTASDPLWEVTIDLPVGTSFEYKFIKVEPSG
TVVWESDPNRQYTVPTACTGTTETVVATWR;
AA-3239 (TauAmy1) from Thermoascus aurantiacus
SEQ ID NO: 33
ATPAQWRSRSVYFLLTDRFARSDGSTTAACDTSARLDYIQ
GMGFTAIWISPVTEQLPQDTGDGTAYHGYWQQDIYSLNPN
FGTADDLRALADALHARGMYLMVDVVANHMGYAGPGNSVD
YSVFNPFNKQEYFHPYCEITNYDDQSNVEDCWLGDTIVSL
PDLNTTRSDVEDIWYSWVRALVSNYSVDGLRIDTVKHVQK
DFWPGYNDAAGVYCVGEVFDGDPSYTCDYQNYLDGVLNYP
MYYPLLRAFSSTSGSISDLYNMINTVKAQCADSTLLGTSG
YSTTSELYQFIAVSNQIRNYAIYVDEGYLTYKAWPIYQDS
HTLAIRKGFDGNQVITVLSNLGSSGSSYTLSLSGTGYAAG
QQVTEIYSCTDVTADSNGNIAVSMGGGLPKAFFPTAKLAG
SGICWK;
AA-3937 (BhaAmy3) from Brevibacterium
halotolerans strain XFB-BI
SEQ ID NO: 34
GPAAANAETQNTSNELTAPSIKSGTILHAWNWSFNTLKHN
MKDIHDAGYTAIQTSPINQVKEGNQGNKSMSNWYWLYQPT
SYQIGNRYLGTEQEFKEMCAAAEEYGVKVIVDAVINHTTS
DYAAISNEIKSIPNWTHGNTQIKNWSDRWDVTQNSLLGLY
DWNTQNTQVQSYLKRFLERALNDGADGFRYDAAKHIELPD
DGNYGSQFWPNITNTSAEFQYGEILQDSASRDAAYANYMN
VTASNYGHSIRSALKNRNLSVSNISHYASEVSADKLVTWV
ESHDTYANDEEESTWMSDDDIRLGWAVIASRSGSTPLFFS
RPEGGGNGVRFPGKSQIGDRGSALFEDQAITAVNRFHNVM
DGQPEELSNPNGNNQIFMNQRGSHGVVLANAGSSSVTINT
STKLPDGRYDNKAGNGSFQVTDGKLTGTINARSVAVLYSD
DIANAPHVFLENVKTGVTHSFNDQLTITLRADANTTKAVY
QINNGQETVFKDGDQLTIGKGDPFGTTYTITLTGTNSDGV
TRTQEYSFVKREPSAAKTIGYQNPNHWGQVNAYIYKHDGG
RALELTGSWPGKAMIKNADGIYTLTLPADTDTTNAKVIFN
NGSAQVPGQNQPGFDYVQNGLYNDSGLSGSLPH;

GA/AA combinations that performed better than TrGA/AkAA are listed in the following table:

| AA | GA |
|---|---|
| AA-1708 | GA-3317 |
| | GA-3298 |
| | GA-2040 |
| | GA-3280 |
| | GA-2441 |
| | GA-1805 |
| | GA-2439 |
| | GA-4686 |
| | GA-3301 |
| | GA-2331 |
| | GA-3275 |
| AA-3238 | GA-3317 |
| | GA-3280 |
| | GA-3298 |
| | GA-4688 |
| | GA-2441 |
| | GA-4686 |
| | GA-2040 |
| AA-2285 | GA-3317 |
| | GA-2441 |
| | GA-3298 |
| | GA-3280 |
| AA-2522 | GA-3317 |
| | GA-3298 |
| | GA-2439 |
| AA-3239 | GA-3298 |
| | GA-1805 |
| | GA-3317 |
| | GA-2439 |
| AA-2303 | GA-3298 |
| | GA-3317 |
| | GA-2439 |
| | GA-3301 |
| | GA-2441 |
| | GA-3280 |
| AA-2940 | GA-3317 |
| AA-1704 | GA-3298 |

In some embodiments, the compositions and methods include a granular starch-converting glucoamylase, or active fragment, thereof, comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99%, amino acid sequence identity to SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, or SEQ ID NO: 20.

In some embodiments, the compositions and methods include a granular starch-converting α-amylase, or active fragment, thereof, comprising an amino acid sequence having at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99%, amino acid sequence identity to SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, or SEQ ID NO: 34.

In some embodiments, the compositions and methods include a granular starch-converting α-amylase having an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to SEQ ID NO: 22, or to an active fragment, thereof, and a granular starch-converting glucoamylase having at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to any one of SEQ ID NOs: 18, 16, 4, 13, 8, 3, 7, 19, 17, 5 or 12, or an active fragments, thereof.

In some embodiments, the compositions and methods include a granular starch-converting α-amylase having an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to SEQ ID NO: 32, or to an active fragment, thereof, and a granular starch-converting glucoamylase having at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to any one of SEQ ID NOs: 18, 13, 16, 20, 8, 19, or 4, or an active fragments, thereof.

In some embodiments, the compositions and methods include a granular starch-converting α-amylase having an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to SEQ ID NO: 25, or to an active fragment, thereof, and a granular starch-converting glucoamylase having at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to any one of SEQ ID NOs: 18, 8, 16, or 13, or an active fragments, thereof.

In some embodiments, the compositions and methods include a granular starch-converting α-amylase having an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to SEQ ID NO: 29, or to an active fragment, thereof, and a granular starch-converting glucoamylase having at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to any one of SEQ ID NOs: 18, 16, or 7, or an active fragments, thereof.

In some embodiments, the compositions and methods include a granular starch-converting α-amylase having an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to SEQ ID NO: 33, or to an active fragment, thereof, and a granular starch-converting glucoamylase having at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to any one of SEQ ID NOs: 16, 3, 18, or 7, or an active fragments, thereof.

In some embodiments, the compositions and methods include a granular starch-converting α-amylase having an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to SEQ ID NO: 27, or to an active fragment, thereof, and a granular starch-converting glucoamylase having at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to any one of SEQ ID NOs: 16, 18, 7, 17, 8, or 13, or an active fragments, thereof.

In some embodiments, the compositions and methods include a granular starch-converting α-amylase having an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to SEQ ID NO: 31, or to an active fragment, thereof, and a granular starch-converting glucoamylase having at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to SEQ ID NO: 18, or an active fragments, thereof.

In some embodiments, the compositions and methods include a granular starch-converting α-amylase having an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to SEQ ID NO: 21, or to an active fragment, thereof, and a granular starch-converting glucoamylase having at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to SEQ ID NO: 16, or an active fragments, thereof.

In some embodiments, the compositions and methods include a plurality of the granular starch-converting glucoamylase and/or α-amylase enzymes described, herein.

In some embodiments, the compositions and methods further include other enzymes, such as other α-amylases and glucoamylases, including other granular starch hydrolyzing enzymes. In some embodiments, the addition enzyme is selected from a cellulase, a glucanase, a xylanase, a phytase, a protease, a trehalase, and a pullulanase.

In some embodiments, the granular starch has a DS of between 5-60%; 10-50%; 15-45%; 15-30%; 20-45%; 20-30% and also 25-40%. The contacting step with glucoamylase and/or α-amylase is conducted at a pH range of 3.0 to 7.0; 3.0 to 6.5; 3 to 5.5; 3.5 to 4.5; 3.5 to 7.0; 3.5 to 6.5; 4.0 to 6.0 or 4.5 to 5.5. The slurry is held in contact at a temperature at or below the starch gelatinization temperature of the granular starch. In some embodiments, this temperature is held between 45° C. and 70° C.; in other embodiments, the temperature is held between 50° C. and 70° C.; between 55° C. and 70° C.; between 60° C. and 70° C., between 60° C. and 65° C.; between 55° C. and 65° C. and between 55° C. and 68° C. In further embodiments, the temperature is at least 45° C., 48° C., 50° C., 53° C., 55° C., 58° C., 60° C., 63° C., 65° C. and 68° C. In other embodiments, the temperature is not greater than 65° C., 68° C., 70° C., 73° C., 75° C. and 80° C.

The initial starch gelatinization temperature ranges for a number of granular starches which may be used in accordance with the processes herein can include, but are not limited to barley (52° C. to 59° C.), wheat (58° C. to 64° C.), rye (57° C. to 70° C.), corn (62° C. to 72° C.), high amylose corn (67° C. to 80° C.), rice (68° C. to 77° C.), sorghum (68° C. to 77° C.), potato (58° C. to 68° C.), tapioca/cassava (59° C. to 69° C.) and sweet potato (58° C. to 72° C.). (J.J.M. Swinkels pg 32-38 in Starch Conversion Technology, Eds Van Beynum et al., (1985) Marcel Dekker Inc. New York and The Alcohol Textbook 3rd ED. A Reference for the Beverage, Fuel and Industrial Alcohol Industries, Eds Jacques et al., (1999) Nottingham University Press, UK).

In the contacting step, the slurry may be held in contact with the present enzymes for a period of 5 minutes to 48 hours; and also for a period of 5 minutes to 24 hours. In some embodiments the period of time is between 15 minutes and 12 hours, 15 minutes and 6 hours, 15 minutes and 4 hours and also 30 minutes and 2 hours. Total ethanol fermentation time typically requires 30-70 hours, for example, 40-70, 30-60, 50-70, 30-50, or similar hours.

During the contacting step between 25-90% or more of the granular starch is solubilized to produce saccharides comprising dextrin, oligosaccharides, and smaller sugars like glucose. In some embodiments, greater than 20%, 25%, 30%, 35%, 40%, 45%, 50%, 55%, 60%, 65%, 70%, 75%, 80%, 85% and 90% of the granular starch is solubilized.

After contacting the granular starch with the α-amylase and glucoamylase for a period of time as indicated above, a soluble starch substrate (mash) is obtained which comprises greater than 10%, 15%, 20%, 25%, 30%, 35%, 40%, 45%, 50%, 55%, 60%, 65%, 70%, 75%, 80%, 90%, 95% and 97% glucose.

After the contacting step which results in the production of a mash comprising glucose, the mash is typically subjected to fermentation with a fermenting microorganism (e.g. an ethanol-producing microorganism). The fermentation can be done simultaneously with the contacting step during which the produced glucose can be converted immediately to the end product by the fermenting microorganism. In this case, the amount of glucose that accumulates in the mash will be much lower, as it is rapidly converted to an end of fermentation product.

In some embodiments the fermenting organism is yeast, optionally recombinant yeast. Examples of yeast include but are not limited to a Saccharomyces sp., a Candida sp., a Pichia sp., a Dekkera sp., an Hanseniaspora sp., a Pseudozyma sp., a Sacharromycodes sp., a Zygosaccharomyces sp., a Zygoascus sp., an Issatchenkia sp., a Williopsis sp., and a Brettanomyces sp. Particular yeast include but are not limited to Saccharomyces cerevisiae, Torulaspora delbrueckii, Brettanomyces bruxellensis, Zygosaccharomyces bailii, Debaryomyces hansenii, and Zygosaccharomyces rouxii.

In some embodiments the fermenting organism is filamentous fungi, optionally recombinant filamentous fungi. Examples of filamentous fungi include but are not limited to a Trichoderma sp., an Aspergillus sp., a Penicillium sp., and a Myceliopthora sp. (such as Cl from Dyadic).

In some embodiments the fermenting organism is a bacterium, optionally a recombinant bacterium. Preferred bacterial fermenting organisms include an Escherichia sp., a Zymomonas sp., a Bacillus sp., a Corynebacterium sp., a Brevibacterium sp., a Streptomyces sp., and a Klebsialla sp. In some embodiments, the bacterium is capable of producing an alcohol, e.g., ethanol, butanol, methanol, propanol etc.

Improved strains of ethanologenic microorganisms, which can withstand higher temperatures, for example, are known in the art and can be used. See Liu et al. (2011) Sheng Wu Gong Cheng Xue Bao 27(7): 1049-56. Commercial sources of yeast include ETHANOL RED® (LeSaffre); THERMOSACC® (Lallemand); RED STAR® (Red Star); FERMIOL® (DSM Specialties); and SUPERSTART® (Alltech).

In some embodiments the fermenting organism expresses enzymes such as the granular starch-converting glucoamylases and/or converting α-amylases described, herein, other glucoamylases and/or α-amylases or starch degrading enzymes, such as pullanase and/or trehalase. Other enzymes include phytase, cellulase, xylanase, glucanase, xylose reductase, xylitol dehydrogenase, protease, and the like.

Use of the present granular starch-converting glucoamylases and α-amylases is not restricted to production of a particular end of fermentation (EOF) product. In some embodiments, the EOF may be, but is not limited to, metabolites, such as citric acid, lactic acid, succinic acid, acetic acid, monosodium glutamate, gluconic acid, sodium gluconate, calcium gluconate, potassium gluconate, itaconic acid and other carboxylic acids, glucono delta-lactone, sodium erythorbate, glutamic acid, tryptophan, threonine, methionine, lysine and other amino acids, omega-3 fatty acid, isoprene, 1,3-propanediol, ethanol, methanol, propanol, butanol, other alcohols, and other biochemicals and biomaterials.

Prior to subjecting the mash to fermentation, the mash may be further exposed to an aqueous solution comprising, for example, backset and/or corn steep, and adjusted to a pH in the range of pH 3.0 to 6.0; pH 3.5 to 5.5, or pH 4.0 to 5.5. In this embodiment of the invention, the % DS of the mash may be diluted. For example, the DS of the diluted mash maybe between 5 to 35%; 5 to 30%; 5 to 25%; 5 to 20%; 5 to 20%; 5 to 15%; and 5 to 10% less than the % DS of the slurry in the contacting step. In one non-limiting example, if the % DS of the slurry in the contacting step is approximately 32% and the mash is further exposed to a diluting aqueous solution which dilutes the DS between 5 to 10%, the DS of the mash to be fermented will be between 22% and 27%. In some specific embodiments, if the DS of the contacting slurry is between 30 to 35%, the DS of the diluted slurry will be about 20 to 30%.

In a specific embodiment, mash comprising at least 10% glucose is then subjected to fermentation processes using fermenting microorganisms as described above. These fermentation processes are described in The Alcohol Textbook 3rd ED, A Reference for the Beverage, Fuel and Industrial Alcohol Industries, Eds Jacques et al., (1999) Nottingham University Press, UK.

In some embodiments, contacting the granular starch with the α-amylase and glucoamylase is performed simultaneously with fermentation by the fermenting microorganism. During this process the glucose content (or that of other fermentable sugars) remains low because it is simultaneously converted to end product by the fermenting microorganisms as described above.

As noted, one EOF product that can be produced using the present compositions and methods is an alcohol product, such as ethanol. The end product produced according to the process may be separated and/or purified from the fermentation media. Methods for separation and purification are known, for example by subjecting the media to extraction, distillation and column chromatography.

In further embodiments, the mash may be separated at any time in fermentation, but preferably at the end of fermentation, and even more preferably after removal of end product ethanol by distillation, by for example centrifugation into the liquid phase and solids phase The alcohol may be recovered by means such as distillation and can be further purified by molecular sieve dehydration or ultra-filtration.

In some embodiments, the yield of ethanol will be greater than 8%, 10%, 12%, 14%, 16% and 18% by volume. The ethanol obtained according to process of the invention may be used as a fuel ethanol, potable ethanol or industrial ethanol.

In addition to the EOF product, the present granular starch-converting glucoamylases and α-amylases may offer advantages in the production or quality of fermentation co-products such as distillers dried grains (DDG) and distiller's dried grain plus solubles (DDGS), which may be used as an animal feed or other applications.

### EXAMPLES

### Example 1

### Evaluation of GA/AA blends in Simultaneous Saccharification and Fermentation

A number of glucoamylases (GA) and α-amylases (AA) were tested in combination as enzyme blends for use in simultaneous saccharification and fermentation using a corn flour substrate.

The GA used are listed in the following table:

| Name | Abbr. | Source organism | SEQ ID NO |
|---|---|---|---|
| GA-1805 | AteGA1 | Aspergillus terreus | 3 |
| GA-2040 | AfuHT3 | Aspergillus fumigatus | 4 |
| GA-2331 | NfiGA1 | Neosartorya fischeri | 5 |
| GA-2437 | AfuGA2 | Neosartorya fumigata | 6 |
| GA-2439 | PmaGA1 | Penicillium marneffei | 7 |
| GA-2441 | TstGA2 | Talaromyces stipitatus | 8 |
| GA-2442 | MacGA1 | Metarhizium acridum | 9 |
| GA-2578 | ScoGA1 | Schizophyllum commune | 10 |
| GA-2722 | Tat GA2 | Trichoderma atroviridis; Hypocrea | 11 |
| | | atroviridis | |
| GA-3275 | BadGA1 | Bjerkandera adusta | 12 |
| GA-3280 | GspGA1 | Ganoderma spp | 13 |
| GA-3283 | TveGA3 | Termetes versicolor | 14 |
| GA-3294 | HsuGA3 | Hypholoma sublateritium | 15 |
| GA-3298 | FmeGA1 | Fomitiporia mediterranea | 16 |
| GA-3301 | PstGA2 | Punctularia strigosozonata | 17 |
| GA-3317 | PbrGA1 | Phlebia brevispora Nakasone | 18 |
| GA-4686 | SzeGA2 | Sarocladium zeae | 19 |
| GA-4688 | GA GO\| | Penicillium oxalicum | 20 |
| | 687 | | |

The AA used are listed in the following table:

| Name | Abbr. | Source organism | SEQ ID NO: |
|---|---|---|---|
| AA-1704 | AcAA | Aspergillus clavatus | 21 |
| AA-1708 | AtAA | Aspergillus terreus | 22 |
| AA-2115 | AfuAmy1 | Aspergillus fumigatus Af293 | 23 |
| AA-2205 | NfiAmy1 | Neosartorya fischeri | 24 |
| AA-2285 | TemAmy1 | Talaromyces emersonii | 25 |
| AA-2301 | PfuAmy1 | Penecillium funiculosum | 26 |
| AA-2303 | PfuAmy3 | Penecillium funiculosum | 27 |
| AA-2506 | ApuAmy1 | Aureobasidium pullulans | 28 |
| AA-2522 | LstAmy1 | Lipomyces starkeyi | 29 |
| AA-2676 | OsaAmy2 | Oryza sativa Japonica Group | 30 |
| AA-2940 | AacAmy2 | Aspergillus aculeatus | 31 |
| AA-3238 | TleAmy1 | Talaromyces leycettanus | 32 |
| AA-3239 | TauAmy1 | Thermoascus_aurantiacus | 33 |
| AA-3937 | BhaAmy3 | Brevibacterium halotolerans | 34 |
| | | strain XFB-BI | |

For the analyses, a slurry of 29.4% dry solids (wt/wt) was made by adding 50%/50% tap water/demineralized water to corn flour substrate (Azure farm Corn Flour organic (FL131)—Azure standard, Dufur Oregon, USA). The pH was adjusted as specified with H2504 and afterwards urea was added to a final concentration of 500 ppm. Finally, 0.001% w/w FERMGEN 2.5xTM protease (DuPont) and 0.1% w/w active dry yeast (Fermentis, France—Ethanol Red) were added. The substrate including the protease and the yeast was divided into the SSF vessels and the selected GA/AA enzyme blend was added (0.107 mg/g ds of GA and 0.016 mg/g ds of AA) to each vessel as well. The vessels were incubated at 32° C. and samples were collected at three different time points (i.e., 24 h, 48 h, and 96 h) to analyze sugar, glycerol, and ethanol content using HPLC.

For Examples 2 and 3, the substrate used in the model system screening was 1% (w/w) corn starch (Sigma, Cat. No. 54126) in 50 mM sodium acetate buffer. α-amylase and glucoamylase were combined at the same protein ratio to that of STARGEN™ 002 (i.e., AkAA:TrGA=1:6.6). For α-amylase (AA) screening, Trichoderma reesei glucoamylase (TrGA; SEQ ID NO: 1) was used as the glucoamylase component and Aspergillus kawachii α-amylase (AkAA) (SEQ ID NO: 2) was the benchmark AA. For glucoamylase screening, AkAA was used as the AA component and TrGA was the benchmark GA. The reaction was initiated by adding 10 μL of glucoamylase and 10 μL of α-amylase to 150 μL of the substrate, with final dosages at 10 ppm and 1.5 ppm for GA and AA, respectively. The incubations were done in iEMS (32° C.; 900 rpm) for 6, 20 and 28 h, respectively. To quench the reaction, 50 μL of 0.5 M NaOH was added and mixed vigorously. The plate was then sealed with a BioRad seal and centrifuged at 2500 rpm for 3 min. For HPLC analysis, the supernatant was diluted by a factor of 10 using 5 mM H2504. The diluted supernatant was filtered and 20 μL of the solution was injected into an Agilent 1200 series HPLC equipped with a refractive index detector. The separation column used was a Phenomenex Rezex-RFQ Fast Fruit column (cat #00D-0223-KO) with a Phenomenex Rezex ROA Organic Acid guard column (cat #03B-0138-KO). The mobile phase was 5 mM H2504, and the flow rate was 1.0 mL/min at 85° C. The amount of glucose released was used to calculate a Performance Index (PI) ration against benchmark AkAA/TrGA combinations.

For Example 4, HPLC (Agilent Technologies 1200 series) run conditions were as follows. A PHENOMENEX REZEX™ RFQ-Fast Acid H+(8%) column was held at 80° C. The solvent was 0.01 N H2SO4 at an isocratic flow of 1.0 ml/min Injection volumes were 10 μl. Runtimes were 5.3 min. Refractive index detection was used to detect DP4+, DP3, DP2, DP1, glycerol, and ethanol. Appropriate calibration standards were used for quantification of the components present.

In all cases, performance indices (PI) relative to a reference blend were calculated with respect to glucose release and/or ethanol production. Performance equal to the reference blend was assigned a PI of 1.0. Blends with a PI greater than 1.0 at any analysis time point or pH are listed in the Tables in the following Examples and represent improvements over current combinations and methods. Unless otherwise specified, all measurement used, herein, are weight/weight (wt/wt; w/w).

### Example 2

### Results obtained using different GA

A number of different GA were individually tested in Aspergillus kawachii α-amylase (AkAA; SEQ ID NO: 2) blends as described in Example 1. The amount of glucose release following 6, 20, and 28 h of incubation at pH 3.5 and 4.5 was measured and divided by the concentration of glucose released by the reference combination of Trichoderma reesei glucoamylase (TrGA; SEQ ID NO: 1) and AkAA. The results for the GA with a PI value greater than 1.0 are shown in the Table, below. 18 GA demonstrated superior performance to TrGA when combined with AkAA, remarkably, in some cases, by two-fold.

| | | pH 3.5 | | | pH 4.5 | | |
|---|---|---|---|---|---|---|---|
| | | PI (AkAA + | | | PI (AkAA + | | |
| | | TrGA = 1.0) | | | TrGA = 1.0) | | |
| AA | GA | 6 h | 20 h | 28 h | 6 h | 20 h | 28 h |
| AkAA | GA-1805 | 1.47 | 1.36 | 1.18 | 1.55 | 1.42 | 1.37 |
| | GA-2040 | 1.67 | 1.4 | 1.23 | 1.64 | 1.43 | 1.41 |
| | GA-2331 | 1.49 | 1.33 | 1.21 | 1.54 | 1.41 | 1.4 |
| | GA-2437 | 1.83 | 1.45 | 1.27 | 1.7 | 1.46 | 1.46 |
| | GA-2439 | 1.94 | 1.49 | 1.28 | 1.97 | 1.56 | 1.52 |
| | GA-2441 | 1.76 | 1.45 | 1.28 | 1.78 | 1.5 | 1.46 |
| | GA-2442 | 1.24 | 1.16 | 1.09 | 1.63 | 1.44 | 1.4 |
| | GA-2578 | 1.84 | 1.47 | 1.28 | 1.58 | 1.46 | 1.37 |
| | GA-2722 | 1.62 | 1.37 | 1.16 | 1.5 | 1.42 | 1.31 |
| | GA-3275 | 1.85 | 1.39 | 1.19 | 1.9 | 1.57 | 1.44 |
| | GA-3280 | 1.86 | 1.42 | 1.21 | 1.66 | 1.48 | 1.4 |
| | GA-3283 | 1.82 | 1.43 | 1.19 | 1.71 | 1.51 | 1.44 |
| | GA-3294 | 1.6 | 1.36 | 1.19 | 1.54 | 1.42 | 1.36 |
| | GA-3298 | 1.86 | 1.46 | 1.23 | 1.65 | 1.47 | 1.39 |
| | GA-3301 | 1.78 | 1.44 | 1.22 | 1.78 | 1.53 | 1.41 |
| | GA-3317 | 2.03 | 1.49 | 1.22 | 1.55 | 1.49 | 1.36 |
| | GA-4686 | 1.6 | 1.36 | 1.19 | 1.67 | 1.45 | 1.36 |
| | GA-4688 | 1.72 | 1.41 | 1.22 | 1.8 | 1.51 | 1.41 |

### Example 3

### Results obtained using different AA

A number of different AA were individually tested in TrGA blends as described in Example 1. The amount of glucose released following 6, 20, and 28 h of incubation at pH 3.5 and 4.5 was measured and divided by the glucose released by the reference combination of TrGA and AkAA. The results for the AA with a PI value greater than 1.0 are shown in the Table, below. Nineteen demonstrated superior performance to AkAA when combined with TrGA, although the improvement was less pronounced than in the case of using different GA in Example 2.

| | | pH 3.5 | | | pH 4.5 | | |
|---|---|---|---|---|---|---|---|
| | | PI (AkAA + | | | PI (AkAA + | | |
| | | TrGA=1.0) | | | TrGA = 1.0) | | |
| GA | AA | 6 h | 20 h | 28 h | 6 h | 20 h | 28 h |
| TrGA | AA-1708 | 1.44 | 1.26 | 1.1 | 1.46 | 1.39 | 1.36 |
| | AA-2115 | 0.91 | 0.58 | 0.52 | 1.44 | 1.37 | 1.33 |
| | AA-2205 | 0.61 | 0.38 | 0.34 | 1.41 | 1.35 | 1.33 |
| | AA-2285 | 1.23 | 1.19 | 1.04 | 1.14 | 1.18 | 1.16 |
| | AA-2301 | 0.9 | 0.72 | 0.67 | 1.3 | 1.26 | 1.17 |
| | AA-2303 | 1.15 | 1.12 | 1.01 | 1.14 | 1.19 | 1.13 |
| | AA-2506 | 1.3 | 1.05 | 0.93 | 1.53 | 1.4 | 1.29 |
| | AA-2522 | 1.14 | 1.06 | 0.97 | 1.08 | 1.08 | 1.05 |
| | AA-2676 | 0.53 | 0.3 | 0.26 | 1.35 | 1.26 | 1.16 |
| | AA-2940 | 1.22 | 1.15 | 0.99 | 1.26 | 1.31 | 1.2 |
| | AA-3238 | 1.27 | 1.22 | 1.13 | 1.31 | 1.35 | 1.26 |
| | AA-3239 | 1.13 | 1 | 0.96 | 1.18 | 1.16 | 1.13 |
| | AA-3937 | 0.79 | 0.41 | 0.36 | 1.47 | 1.42 | 1.34 |
| | AA-1704 | 1.24 | 1.1 | 1.09 | 1.35 | 1.3 | 1.25 |
| | AKAA | 1 | 1 | 1 | 1 | 1 | 1 |

### Example 4

### Identification of high performing GA/AA blends

A number of different AA/GA blends were tested as described in Example 1. The concentration of ethanol following 24, 48, and 96 h of incubation at pH 3.5 was measured, averaged, and divided by the concentration of ethanol produced by the reference combination of TrGA and AkAA.

Blends with a PI greater than 1.0 are listed in the following Table.

| AA | GA | PI |
|---|---|---|
| AA-1708 | GA-3317 | 1.32 |
| | GA-3298 | 1.3 |
| | GA-2040 | 1.22 |
| | GA-3280 | 1.21 |
| | GA-2441 | 1.2 |
| | GA-1805 | 1.2 |
| | GA-2439 | 1.18 |
| | GA-4686 | 1.15 |
| | GA-3301 | 1.15 |
| | GA-2331 | 1.14 |
| | GA-3275 | 1.11 |
| AA-3238 | GA-3317 | 1.19 |
| | GA-3280 | 1.17 |
| | GA-3298 | 1.16 |
| | GA-4688 | 1.16 |
| | GA-2441 | 1.15 |
| | GA-4686 | 1.11 |
| | GA-2040 | 1.10 |
| AA-2285 | GA-3317 | 1.14 |
| | GA-2441 | 1.13 |
| | GA-3298 | 1.12 |
| | GA-3280 | 1.10 |
| AA-2522 | GA-3317 | 1.11 |
| | GA-3298 | 1.10 |
| | GA-2439 | 1.10 |
| AA-3239 | GA-3298 | 1.11 |
| | GA-1805 | 1.10 |
| | GA-3317 | 1.10 |
| | GA-2439 | 1.08 |
| AA-2303 | GA-3298 | 1.15 |
| | GA-3317 | 1.14 |
| | GA-2439 | 1.13 |
| | GA-3301 | 1.12 |
| | GA-2441 | 1.10 |
| | GA-3280 | 1.08 |
| AA-2940 | GA-3317 | 1.12 |
| AA-1704 | GA-3298 | 1.08 |

All patents and publications, including all sequences disclosed within such patents and publications, referred to herein are expressly incorporated by reference in their entirety for all purposes. Insofar as the product referred to by a trademark name varies with time, the product having the characteristics described in the relevant product literature, including websites, from the manufacturer as of the effective filing date of the application is intended. Such product literature is also incorporated by reference in its entirety for all purposes. The headings provided herein are not limitations of the various aspects or embodiments of the invention, which can be had by reference to the specification as a whole. Although preferred methods and materials have been described, any methods and materials similar or equivalent to those described herein can be used in the practice or testing of the present invention. Unless otherwise apparent from the context, any embodiment, aspect, step, feature, element or limitation can be used in combination with any other.

1. A method for processing granular starch comprising:
contacting a slurry comprising granular starch with a glucoamylase and a granular starch-converting α-amylase, at a temperature at or below the gelatinization temperature of the granular starch, to produce saccharides fermentable by a fermenting organism; wherein the granular starch-converting α-amylase comprises an amino acid sequence having at least 85% amino acid sequence identity to any one of SEQ ID NOs:22, 27 or 32, or at least 85% amino acid sequence identity to an active fragment, thereof.

2. The method of claim 1, wherein contacting the slurry with the glucoamylase and the granular starch-converting α-amylase results in increased starch conversion compared to contacting the same slurry with the same glucoamylase and α-amylase from Aspergillus kawachii (AkAA) having the amino acid sequence of SEQ ID NO: 2.

3. The method of claim 1, wherein contacting the slurry with the glucoamylase and the granular starch-converting α-amylase results in increased glucose release compared to contacting the same slurry with the same glucoamylase and α-amylase from Aspergillus kawachii (AkAA) having the amino acid sequence of SEQ ID NO: 2.

4. The method of claim 1, wherein contacting the slurry with the glucoamylase and the granular starch-converting α-amylase results in increased total glucose equivalents compared to contacting the same slurry with the same glucoamylase and α-amylase from Aspergillus kawachii (AkAA) having the amino acid sequence of SEQ ID NO: 2.

5. The method of claim 4, wherein the increased total glucose equivalents is at least 5% higher, and preferably at least 10% higher, compared to the amount produced by contacting the same slurry with the glucoamylase and α-amylase from Aspergillus kawachii (AkAA) having the amino acid sequence of SEQ ID NO: 2.

6. The method of claim 1, wherein the method results in the production of glucose, maltose, oligosaccharides, or a mixture thereof, optionally in the form of a syrup.

7. The method of claim 1, further comprising contacting the saccharides with a fermenting organism to produce an end of fermentation product; wherein the contacting results in increased production of an end of fermentation product compared to contacting the same slurry with the glucoamylase and α-amylase from Aspergillus kawachii (AkAA) having the amino acid sequence of SEQ ID NO: 2.

8. The method of claim 7, wherein the end of fermentation product is ethanol.

9. The method of claim 7, wherein the end of fermentation product is a non-ethanol biochemical.

10. The method of claim 1, wherein the glucoamylase and the granular starch-converting α-amylase are added simultaneously.

11. The method of fief claim 7, wherein the glucoamylase and the granular starch-converting α-amylase and the fermenting organism are added simultaneously.

12. The method of claim 1, wherein the glucoamylase and the granular starch-converting α-amylase are produced by a fermenting organism.

13. The method of claim 1, further comprising the addition of an additional enzyme to the slurry.

14. The method of claim 1, wherein the glucoamylase has at least 85% amino acid sequence identity to a glucoamylase selected from the group consisting of SEQ ID NOs: 1 and 3-20, or to an active fragment, thereof.

15. (canceled)

16. A granular starch-converting α-amylase comprising an amino acid sequence having at least 85% amino acid sequence identity to any one of SEQ ID NOs: 22, 27 or 32, or at least 85% amino acid sequence identity to an active fragment, thereof; wherein the granular starch-converting α-amylase, upon contacting a slurry of granular starch in combination with a glucoamylase, is capable of increased starch conversion, increased glucose release, and/or the production of increased total glucose equivalents, compared to contacting the same slurry with the same glucoamylase and α-amylase from Aspergillus kawachii (AkAA) having the amino acid sequence of SEQ ID NO: 2.

17. The starch-converting α-amylase of claim 16, wherein the granular starch-converting α-amylase, upon contacting a slurry of granular starch in combination with a glucoamylase, is capable of at least 5% higher, and preferably at least 10% higher, production of increased total glucose equivalents compared to contacting the same slurry with the same glucoamylase and α-amylase from Aspergillus kawachii (AkAA) having the amino acid sequence of SEQ ID NO: 2.

18. The granular starch-converting α-amylase of claim 16, wherein the granular starch-converting α-amylase, upon contacting a slurry of granular starch in combination with a glucoamylase and a fermenting organism, is capable of increased production of an end of fermentation product compared to contacting the same slurry with the same glucoamylase and α-amylase from Aspergillus kawachii (AkAA) having the amino acid sequence of SEQ ID NO: 2.

19. A composition comprising the granular starch-converting α-amylase of claim 16 in combination with a glucoamylase.

20. The composition of claim 19, wherein the glucoamylase has at least 85% amino acid sequence identity to an α-amylase selected from the group consisting of SEQ ID NOs: 1 and 3-20, or to an active fragment, thereof.

21. (canceled)

22. A fermenting organism capable of producing the granular starch-converting α-amylase of claim 16, optionally in combination with a glucoamylase, which glucoamylase may optionally be selected from claim 20.

23. The method of claim 7, wherein the glucoamylase or the granular starch-converting α-amylase and the fermenting organism are added simultaneously.

24. The method of claim 1, wherein the glucoamylase or the granular starch-converting α-amylase are produced by a fermenting organism.
